(19) United States
(12) Patent Application Publication
(10) Pub. No.: US 2019/0180877 A1
(43) Pub. Date: Jun. 13, 2019

(54) ESTROGEN METABOLITE LEVELS AND CYP1B1 POLYMORPHISMS IN LUNG CANCER DIAGNOSIS, PROGNOSIS, AND RISK ASSESSMENT

(71) Applicant: Institute For Cancer Research d/b/a The Research Institute Of Fox Chase Cancer Center, Philadelphia, PA (US)

(72) Inventors: Margie Clapper, Philadelphia, PA (US); Jing Peng, Philadelphia, PA (US)

(21) Appl. No.: 16/106,761

(22) Filed: Aug. 21, 2018

Related U.S. Application Data
(63) Continuation of application No. 14/468,862, filed on Aug. 26, 2014, now abandoned.
Application No. 14/468,862 is a continuation of application No. PCT/US2013/027716, filed on Feb. 26, 2013.
(60) Provisional application No. 61/603,611, filed on Feb. 27, 2012.

(51) Int. Cl. G16H 50/30; G16B 30/00; C12Q 1/6886

(52) U.S. Cl. CPC G16H 50/30; G16B 30/00; C12Q 2600/156; C12Q 2600/118; C12Q 1/6886

(57) ABSTRACT

Systems and methods for determining the prognosis of a patient having CYP1B1-mediated lung cancer and for diagnosing a risk of developing CYP1B1-mediated lung cancer are provided. The systems and methods comprise determinations of the concentration of estrogen metabolites in the lung tissue or a proxy thereof, or polymorphisms in the gene encoding the CYP1B1 protein, which metabolite concentrations or CYP1B1 polymorphisms are associated with a probability of surviving and/or a risk of developing lung cancer.

### CROSS REFERENCE TO RELATED APPLICATIONS

This application is a continuation of International Application No. PCT/US2013/027716, filed on Feb. 26, 2013, which claims priority to U.S. Provisional Application No. 61/603,611, filed on Feb. 27, 2012, the contents of each are incorporated by reference herein, in their entirety and for all purposes.

### STATEMENT OF GOVERNMENT SUPPORT

The inventions described herein were made, in part, with funds obtained from the National Cancer Institute, Grant No. CA-006927. The U.S. government may have certain rights in these inventions.

### REFERENCE TO A SEQUENCE LISTING

This application includes a Sequence Listing submitted electronically as a text file named Estrogen_Metabolite_ST25.txt, created on Feb. 21, 2013 with a size of 36,000 bytes. The Sequence Listing is incorporated by reference herein.

### FIELD OF THE INVENTION

The invention relates generally to the field of cancer diagnosis, prognosis, and risk assessment. More particularly, the invention relates to systems and methods for evaluating polymorphisms in the CYP1B1 gene and estrogen metabolite levels that correlate with a probability of being at risk for or having lung cancer, and the prognosis of a subject diagnosed with lung cancer.

### BACKGROUND OF THE INVENTION

Various publications, including patents, published applications, technical articles, scholarly articles, and polynucleotide/polypeptide accession numbers are cited throughout the specification. Each of these cited publications is incorporated by reference herein, in its entirety and for all purposes.

Lung cancer is the leading cause of cancer death among men and women in the U.S. In addition to cigarette smoke, estrogen exposure has been associated recently with lung cancer in women. The use of hormone replacement therapy has been related to both a younger age of diagnosis of lung cancer and decreased median survival. Metabolism and detoxification of the constituents of cigarette smoke play a role in lung carcinogenesis. In addition, the activity and carcinogenicity of estrogen depend on the metabolic transformation of 17β-estradiol. It is believed that the balance between the activity of Phase I and II metabolism enzymes affects cell protection from carcinogens and plays an important role in lung carcinogenesis.

Considerable inter-individual genetic variability exists in the Phase I and II enzymes, with several studies suggesting that select polymorphisms are associated with an increased risk for lung cancer development. Nebert D W et al, (2006) Nat. Rev. Cancer 6:947-60. Members of the cytochrome P450 family 1, including CYP1A1 and CYP1B1, activate exogenous substances such as polycyclic aromatic hydrocarbons as well as endogenous substances such as estrogens to highly reactive intermediates. Phase II metabolism enzymes, including glutathione S-transferase M1 (GSTM1), are, in general, responsible for the conversion of these intermediates to inactive conjugates. Previous reports suggested that the replacement of isoleucine with valine at codon 462 of CYP1A1 (1462V) combined with deletion of the GSTM1 gene confer an increased risk of lung cancer in females (odds ratio (OR) 6.54; 95% confidence interval (95% CI) 1.07-40.00). Dresler C et al. (2000) Lung Cancer 30:153-60. The 1462V polymorphism leads to enhanced CYP1A1 enzyme activity, promoting carcinogen activation, while deletion of the GSTM1 gene impairs one's capacity to conjugate and eliminate carcinogens.

CYP1B1 is the predominant enzyme that catalyzes the 4-hydroxylation of estrogen to its most carcinogenic metabolite. Polymorphisms within the coding region of CYP1B1 (codons 48, 119, 432 and 453) have been identified, and the haplotypes containing the variant alleles have been denoted as CYP1B1*2 (48 and 119), *3 (432) and *4 (453), according to the Human Cytochrome P450 Allele Nomenclature Committee. A leucine to valine substitution at codon 432, which confers increased catalytic activity, has been associated with increased risk for lung, prostate, ovarian, renal, and breast cancer as well as head and neck cancer. However, the effect of combined polymorphisms in codons 48, 119, 432 and 453 of CYP1B1 on either susceptibility for lung cancer or patient survival has not been evaluated to date.

The outcome of patients with lung cancer varies widely depending on individual variables including tumor type, stage at presentation, smoking status and gender. For instance, the 5-year survival of lung cancer patients who are current smokers is significantly lower than that of lung cancer patients who never smoked (16% and 23%, respectively, p=0.004). Recent pharmacogenetic studies have also found that polymorphisms in DNA repair enzymes impact the outcome of lung cancer patients treated with specific chemotherapeutic agents.

There is a need in the art to be able to enhance the confidence in prognostic and/or diagnostic information provided to patients, including the assessment of a patient's risk of developing a disease or condition and the identification of a need for preventive intervention. Related to this, there is a need for information that can assist medical practitioners in diagnosing patients, considering treatment regimens, and in determining a patient's prognosis.

### SUMMARY OF THE INVENTION

A method for diagnosing a risk of developing lung cancer comprises determining the concentration of one or more estrogen metabolites in a tissue sample obtained from a subject, comparing the determined concentration with one or more metabolite reference concentrations for a healthy subject, metabolite reference concentrations for a subject at risk for developing lung cancer, or metabolite reference concentrations for a subject having lung cancer, and determining whether the subject is healthy, is at risk for developing lung cancer, or has lung cancer based on the comparison. Preferably, the comparing is carried out using a processor programmed to compare determined concentrations and metabolite reference concentrations. The method may further comprise determining the concentration of one or more estrogens in the tissue sample, comparing the determined concentration with one or more estrogen reference concentrations for a healthy subject, estrogen reference concentrations for a subject at risk for developing lung cancer, or estrogen reference concentrations for a subject having lung cancer, and determining whether the subject is healthy, is at risk for developing lung cancer, or has lung cancer based on the comparison of the determined estrogen metabolite concentrations with metabolite reference concentrations and the determined estrogen concentrations with estrogen reference concentrations. The methods may further comprise determining the prognosis of the subject if the subject is determined to have lung cancer.

The one or more estrogen metabolites may comprise a metabolite produced by the biologic activity of CYP1B1. The one or more estrogen metabolites may be selected from the group consisting of 2-OHE₁, 2-OHE₂, 4-OHE₂, 16-alpha-OHE₁, 2-OMeE₁, 2-OMeE₂, 2-hydroxyestrone-3-methyl ether, 4-OMeE₁, 4-OMeE₂, 17-epiestriol, 16-ketoestradiol, and 16-epiestriol. The one or more estrogens may be E₁, E₂, or E₃. The tissue may comprise one or more of lung tissue, blood, and/or buccal tissue. The tissue may comprise intrathoracic tissue from the bronchus or the lung. The tissue may comprise extrathoracic tissue, for example, tissue from the mouth or the nose as surrogates for intrathoracic tissue because they share a similar gene expression signature. The tissue may comprise blood.

Methods for determining the prognosis of a subject diagnosed with lung cancer comprise determining the sequence of a nucleic acid encoding the CYP1B1 protein in a tissue sample obtained from a subject, comparing the determined sequence with one or more reference sequences using a processor programmed to compare determined sequences and reference sequences, and determining the subject's prognosis based on the comparison. The reference sequences comprise one or more nucleic acid sequences comprising one or more alterations in the wild type CYP1B1 nucleic acid sequence associated with a probability of surviving lung cancer, for example, lung cancer caused by tobacco smoke exposure, and optionally, a wild type CYP1B1 nucleic acid sequence. The one or more alterations may comprise a polymorphism.

Methods for determining the prognosis of a subject diagnosed with lung cancer may also comprise contacting a nucleic acid encoding the CYP1B1 protein in a tissue sample obtained from a subject with one or more polynucleotide probes having a nucleic acid sequence complementary to a CYP1B1 nucleic acid sequence having one or more alterations associated with a probability of surviving lung cancer, including alterations caused by tobacco smoke exposure, and optionally, also with one or more reference probes having a nucleic acid sequence complementary to a wild type CYP1B1 nucleic acid sequence, determining whether the one or more probes, and optionally, whether the one or more reference probes, have hybridized with the nucleic acid, and determining the subject's prognosis based on the determination of whether the probes have hybridized with the nucleic acid. It is preferred that the one or more polynucleotide probes hybridize under stringent conditions to the CYP1B1 nucleic acid sequence. The methods may further comprise identifying which of the probes hybridized with the nucleic acid if more than one probe was contacted with the nucleic acid. The one or more alterations may comprise a polymorphism.

A polymorphism may occur at the position corresponding to codon 48 of CYP1B1 cDNA, at the position corresponding to codon 119 of CYP1B1 cDNA, at the position corresponding to codon 48 and at the position corresponding to codon 119 of CYP1B1 cDNA, at the position corresponding to codon 432 of CYP1B1 cDNA, or at the position corresponding to codon 453 of CYP1B1 cDNA. The polymorphism at the position corresponding to codon 48 may encode a glycine residue. The polymorphism at the position corresponding to codon 119 may encode a serine residue. The polymorphism at the position corresponding to codon 432 may encode a valine residue. The polymorphism at the position corresponding to codon 453 may encode a serine residue. Optionally, the methods may comprise determining whether genomic DNA encoding the CYP1B1 protein obtained from the subject is homozygous for the codon CTG at the position corresponding to codon 432 of CYP1B1 cDNA, if it is determined that the nucleic acid encoding the CYP1B1 protein in the tissue sample has the codon CTG at the position corresponding to codon 432 of CYP1B1 cDNA.

The methods optionally may comprise treating the subject with a regimen capable of improving the prognosis of a lung cancer patient. The regimen may comprise one or more of surgery, radiation therapy, proton therapy, ablation therapy, hormone therapy, chemotherapy, immunotherapy, stem cell therapy, follow up testing, diet management, vitamin supplementation, nutritional supplementation, exercise, physical therapy, prosthetics, kidney transplantation, reconstruction, psychological counseling, social counseling, education, and regimen compliance management.

A system for diagnosing a risk of developing lung cancer comprises a data structure comprising one or more reference concentrations for an estrogen metabolite. The data structure may comprise one or more reference concentrations for an estrogen hormone. The reference concentrations for an estrogen metabolite, and the reference concentrations for an estrogen hormone, comprise concentrations that indicate a subject is at risk for developing lung cancer, concentrations that indicate the subject has lung cancer, and concentrations that indicate the subject is not at risk for developing lung cancer. A processor preferably is operably connected to the data structure, and the processor is preferably programmed to compare determined estrogen metabolite concentrations with reference concentrations for an estrogen metabolite, and the processor is preferably programmed to compare determined estrogen concentrations with reference concentrations for an estrogen hormone, and the processor is preferably programmed to generate a lung cancer development risk assessment based on the comparison of determined concentrations (metabolite and/or estrogen) with reference concentrations (metabolite and/or estrogen). The system may further comprise a system for determining the prognosis of a lung cancer subject.

A system for determining the prognosis of a lung cancer subject comprises a data structure comprising one or more reference nucleic acid sequences having one or more alterations in the wild type CYP1B1 sequence associated with a probability of surviving lung cancer, for example, lung cancer caused by tobacco smoke exposure, and optionally comprising one or more reference nucleic acid sequences having the wild type CYP1B1 nucleic acid sequence, and a processor operably connected to the data structure. The system may exist independently of a system for diagnosing a risk for developing lung cancer. Preferably, the processor is capable of comparing the sequence of a nucleic acid encoding the CYP1B1 protein determined from a tissue sample obtained from a subject with the reference nucleic acid sequences and the wild type reference nucleic acid sequences. The system may further comprise a processor capable of determining the sequence of a nucleic acid encoding the CYP1B1 protein in a tissue sample obtained from the subject. The system may further comprise an input for accepting the determined sequence of the nucleic acid encoding the CYP1B1 protein obtained from the subject. The system may further comprise executable code for causing a programmable processor to determine a prognosis of a lung cancer subject from a comparison of the determined nucleic acid sequence with the reference nucleic acid sequences. The system may further comprise an output for providing results of the comparison to a user.

Computer-readable media may comprise executable code for causing a programmable processor to compare estrogen metabolite, and optionally, estrogen concentrations determined from a tissue sample isolated from a subject with reference concentrations for an estrogen metabolite, and optionally, for an estrogen hormone, that indicate a subject is at risk for developing lung cancer, that indicate the subject has lung cancer, and that indicate the subject is not at risk for developing lung cancer, and comprising executable code for causing a programmable processor to generate a lung cancer development risk assessment based on the comparison of determined concentrations with reference concentrations. The media may further comprise executable code for causing a programmable processor to compare nucleic acid sequences.

Computer-readable media may comprise executable code for causing a programmable processor to compare the sequence of a nucleic acid encoding the CYP1B1 protein determined from a tissue sample obtained from a subject with one or more reference nucleic acid sequences having one or more alterations in the wild type nucleic acid sequence encoding the CYP1B1 protein associated with a probability of surviving lung cancer, for example, lung cancer caused by tobacco smoke exposure, and optionally with one or more wild type reference nucleic acid sequences having the wild type sequence encoding the CYP1B1 protein. The executable code may exist independently of executable code that causes a processor to compare estrogen and estrogen metabolite concentrations. The computer-readable media may further comprise a processor. The computer-readable media may further comprise executable code for causing a programmable processor to determine a prognosis of a lung cancer subject from a comparison of the determined nucleic acid sequence with the reference nucleic acid sequences.

### BRIEF DESCRIPTION OF THE DRAWINGS

FIG. 1 shows Kaplan-Meier curves of the overall survival of lung cancer patients according to a polymorphism at codon 48 of CYP1B1. Univariate survival analyses for CYP1B1 were stratified by gender (top panels) and pack-years of smoking (bottom panels). The p values represent the comparison between homozygous variant (GG) and the combined CC and CG genotypes. The number of individuals (N) and the survival rate at 5 years of follow-up (parentheses) are indicated for each category.

FIG. 2 shows the level of estrogen and its metabolites within the lungs of 129SvJ mice as determined by liquid chromatography/tandem mass spectrometry (LC-MS²). Panel A shows comparison of levels by gender. The level of 4-OH (Panel B), 2-OH (Panel C) and 2-OMe (Panel D) metabolites are expressed as a percentage of total estrogen (summary of all estrogens and metabolites). Values represent the mean±SD (n=5). Asterisks indicate statistical significance (P≤0.05) based on a two-sided Mann-Whitney-Wilcoxon test.

FIG. 3A and FIG. 3B show a gender comparison of total lung tumor burden (total tumor volume) as measured by MRI, in LSL-KrasG12D mice. FIG. 3A shows lung tumors in female mice grow ^{˜}1.2 fold faster than in males. Statistical analysis showed borderline significance (p=0.056, by a two-sided Wald test of the coefficients associated with a unit increase in Ln-transformed total tumor volume over time). FIG. 3B shows females tend to have higher total tumor burden 16 weeks after AdeCre infection. Red lines indicate the medians of each group (p=0.069 by a two-sided Mann-Whitney-Wilcoxon test).

FIG. 4 shows the impact of Cyp1b1 deletion on estrogen metabolism. Panel A shows a comparison of estrogens and EM levels within the lungs of 129SvJ Cyp1b1-WT and Cyp1b1-KO mice as determined by LC-MS2. Asterisks indicate the metabolites levels that differ significantly in WT and KO mice. Panels B and C show expression of Cyp1a1 and Comt in the lungs of Cyp1b1-WT and Cyp1b1-KO mice. The transcript level of each gene was determined by quantitative RT-PCR and normalized against that of the housekeeping gene Hprt. The fold difference was calculated using the ΔΔCt method. The levels for WT female mice have been set arbitrarily to 1. Values represent the mean±SEM (n=5 females and 4 males). *P≤0.05, **P≤0.01.

FIG. 5 shows a comparison of estrogens and EM levels within the lungs of female versus male Cyp1b1-KO mice. Panel A shows absolute levels of each metabolite species. Panels B, C, and D show levels of 4-OH, 2-OH and 2-OMe metabolites expressed as a percentage of total estrogen (sum of all estrogens and EM). Values represent the mean±SEM (n=5 females and 4 males). Asterisks indicate the EM whose levels are significantly different in males and females. *P≤0.05.

FIG. 6 shows the effect of tobacco smoke on the expression of key estrogen-metabolizing genes (panels A, B, and C). The transcript level of each gene was determined by quantitative RT-PCR

and normalized against that of the housekeeping gene Hprt. The fold difference was calculated using the ΔΔCt method. The levels for control mice have been set arbitrarily to 1. Values are expressed as the mean±SEM (n=5 per group). *P≤0.05.

FIG. 7 shows estrogen metabolite levels are modulated by tobacco smoke exposure. Panel A shows a comparison of levels in the lungs of mice exposed to either air (control) or tobacco smoke (smoked). The level of 4-OH (Panel B), 2-OH (Panel C) and 2-OMe (Panel D) metabolites are expressed as a percentage of total estrogen (summary of all estrogens and metabolites). Values represent the mean±SD (2 pools of 5 mice/group). Asterisks indicate statistical significance (P≤0.05) based on a two-sided Student's t-test.

FIG. 8A and FIG. 8B show estrogen metabolite levels in human female lung cancer patients. FIG. 8A shows levels of three estrogens and six estrogen metabolites detected in human lung tissue. FIG. 8B shows the level of each estrogen or estrogen metabolite is higher in tumors compared to adjacent normal tissues (p<0.05 based on signed-rank Wilcoxon tests, n=9). Estrogen (sum of E₁, E₂, and E₃) and 4-OHEs are increased approximately two-fold, while 2-OHEs and 2-OMEs are increased about 1.5-fold and 1.2-fold, respectively.

FIG. 9 shows estrogen metabolite levels are modulated by tobacco smoke exposure (human lungs) (estrogens E₁-E₃, upper left panel, 4-OHEs, upper right panel, 2OHEs, lower left panel, and 2-OMEs, lower right panel). 4-OHEs are higher in the non-neoplastic lung tissue of smokers (S) (n=5) as compared to non-smokers (NS) (n=4) based on Mann-Whitney-Wilcoxon tests.

### DETAILED DESCRIPTION OF THE INVENTION

Various terms relating to aspects of the invention are used throughout the specification and claims. Such terms are to be given their ordinary meaning in the art, unless otherwise indicated. Other specifically defined terms are to be construed in a manner consistent with the definition provided herein.

As used herein, the singular forms “a,” “an,” and “the,” include plural referents unless expressly stated otherwise.

The terms subject or patient are used interchangeably.

Nucleic acid molecules include any chain of at least two nucleotides, which may be unmodified or modified RNA or DNA, hybrids of RNA and DNA, and may be single, double, or triple stranded.

It has been observed in accordance with the invention that levels of estrogen metabolites in both mouse and human lung tissue are modulated based upon exposure to tobacco smoke. These modulations were observed to correlate with the probability of developing lung cancer, particularly as a result of tobacco smoke exposure. Without intending to be limited to any particular theory or mechanism of action, it is believed that modulation of estrogen metabolite levels in the lung result from the activity of CYP1B1, including variants of CYP1B1 encoded by genes having certain polymorphisms. Accordingly, the invention features various methods for characterizing the likelihood of a subject developing and/or surviving lung cancer from tobacco smoke exposure. The methods may be carried out in vivo, in situ, or in vitro.

In some aspects, the methods are diagnostic methods, including methods for determining a risk of developing lung cancer, particularly upon exposure to tobacco smoke. Thus, in one aspect, the invention features methods for diagnosing a risk of developing lung cancer, including lung cancer caused by tobacco smoke exposure in a subject, which relate to measuring levels of estrogen metabolites in tissue samples obtained from the subject. The estrogen metabolites may be those generated by the biologic activity of CYP1B1, or a variant of CYP1B1 encoded by a polymorphism such as those polymorphisms described or exemplified herein. In general, the methods comprise determining the concentration of one or more estrogen metabolites in a tissue sample obtained from a subject, comparing the determined concentration with one or more estrogen metabolite reference concentrations for a healthy subject, estrogen metabolite reference concentrations for a subject at risk for developing lung cancer, or estrogen metabolite reference concentrations for a subject having lung cancer, and determining whether the subject is healthy, is at risk for developing lung cancer, or has lung cancer based on the comparison. The comparing step may be carried out using a processor programmed to compare determined estrogen metabolite concentrations (obtained from the subject) with estrogen metabolite reference concentrations.

The tobacco smoke exposure may be that of a non-smoker, or a present or former light smoker, moderate smoker, or heavy smoker. The tobacco smoke exposure may be that of a non-smoker exposed to second-hand tobacco smoke, including low, moderate, or high levels of second-hand tobacco smoke. The tobacco smoke source may be that of a cigarette, pipe, cigar, or other tobacco product that is burned and inhaled.

The risk may be general and open-ended, for example, a risk of developing lung cancer at some point during the subject's life, for example, assuming no significant change in the current levels of tobacco smoke exposure, or assuming a significant change in the level of exposure, either lower exposure or higher exposure. The risk may be for a former smoker, and may relate to the period of time since the subject stopped smoking tobacco, for example, about six months, about one year, about 1.5 years, about 2 years, about 2.5 years, about 3 years, about 4 years, about 5 years, about 6 years, about 7 years, about 8 years, about 9 years, about 10 years, about 12 years, about 15 years, about 17 years, about 20 years, or more since the subject stopped smoking tobacco. The risk may relate to a particular temporal period or range, for example, a risk of developing lung cancer within about six months, about one year, about 1.5 years, about 2 years, about 2.5 years, about 3 years, about 4 years, about 5 years, about 6 years, about 7 years, about 8 years, about 9 years, about 10 years, about 12 years, about 15 years, about 17 years, about 20 years, or more. A temporal range may be, for example, about 3 to about 6 months, about 6 months to about 1 year, about 1 year to about 1.5 years, about 1 year to about 2 years, about 1 year to about 3 years, about 1 year to about 5 years, about 2 years to about 3 years, about 2 years to about 4 years, about 2 years to about 5 years, about 3 years to about 4 years, about 3 years to about 5 years, about 5 years to about 10 years, about 5 years to about 15 years, about 10 years to about 15 years, about 10 years to about 20 years, or about 10 years to about 25 years. Any temporal period may be based on assuming no significant change in the current levels of tobacco smoke exposure, or assuming a significant change in the level of exposure, either lower exposure or higher exposure. The risk may be a negligible risk, a low risk, a moderately low risk, a moderate risk, a moderately high risk, a high risk, or severe risk. The risk may comprise a risk score, for example, a numerical score on the scale of 0 to 10, including fractions thereof, with 0 representing the lowest or highest risk and with 10 representing the corresponding highest or lowest risk. Such a risk score may arise, for example, according to population studies carried out over time.

In some aspects, the methods further comprise determining the concentration of one or more estrogens in a tissue sample obtained from the subject (which may be the same tissue sample from which the metabolite concentrations were determined, or may be a second tissue sample), comparing the determined concentration with one or more estrogen reference concentrations for a healthy subject, estrogen reference concentrations for a subject at risk for developing lung cancer, or estrogen reference concentrations for a subject having lung cancer, and determining whether the subject is healthy, is at risk for developing lung cancer, or has lung cancer based on the comparison of both the determined estrogen metabolite concentrations with metabolite reference concentrations and the determined estrogen concentrations with estrogen reference concentrations. The comparing step may be carried out using a processor programmed to compare determined estrogen concentrations with estrogen reference concentrations, and estrogen metabolite concentrations with estrogen metabolite reference concentrations.

The one or more estrogen metabolites may comprise one or more of 2-OHE₁, 4-OHE₁, 4-OHE₂, 16-alpha-OHE₁, 2-OMeE₁, 2-OMeE₂ and 2-OHE₁, 2-OHE₂, 2-hydroxyestrone-3-methyl ether, 4-OMeE₁, 4-OMeE₂, 17-epiestriol, 16-ketoestradiol, and/or 16-epiestriol. (Fuhrman B J et al. (2012) J. Natl. Cancer Inst. 104:1-14, and, Eliassen A H et al. (2012) Cancer Res. 72(3):696-706). The estrogen may comprise estrone (E₁), estradiol (E₂), and/or estriol (E₃). The metabolites may be produced by the biologic activity of CYP1B1, or a variant thereof such as a variant encoded by a CYP1B1 gene polymorphism, including those described or exemplified herein.

The tissue may comprise any tissue in which estrogen and/or estrogen metabolites may be found, and in which concentrations of each may be determined. In some aspects, the tissue is lung tissue. In some aspects, tissue from the aerodigestive tract may be used. In some aspects, the tissue is buccal tissue. In some aspects, the tissue is blood. In some aspects, the tissue comprises blood. The tissue may comprise intrathoracic tissue or cells obtained from the bronchus or lung, or may comprise extrathoracic tissue from the mouth or nose, which share a similar gene expression signature. See, e.g., Sridhar S et al. (2008) BMC Genomics 9:259, and Boyle J O et al. (2010) Cancer Prev. Res. 3:266-78. The tissue may comprise a biologic fluid, including blood, mucus, sputum, urine, saliva, tears, and other fluids in which estrogen metabolites may be present and may correlate with a lung cancer risk. Tissue samples may be obtained according to any suitable technique.

The steps of the methods, including any optional steps, may be repeated after a period of time, for example, as a way to monitor a subject's health and prognosis. Thus for example, in some aspects, the methods optionally further comprise repeating the determining and comparing steps after a period of time. Repeating the methods may be used, for example, to determine if a subject has advanced from a healthy state to a precancerous or cancerous state. Repeating the methods may be used, for example, to determine if the patient's prognosis has improved based on a particular treatment regimen, or to determine if adjustments to the treatment regimen should be made to achieve improvement or to attain further improvement in the patient's prognosis. The methods may be repeated at least one time, two times, three times, four times, or five or more times. The methods may be repeated as often as the patient desires, or is willing or able to participate.

The period of time between repeats may vary, and may be regular or irregular. In some aspects, the methods are repeated in three month intervals. In some aspects, the methods are repeated in six month intervals. In some aspects, the methods are repeated in one year intervals. In some aspects, the methods are repeated in two year intervals. In some aspects, the methods are repeated in five year intervals. In some aspects, the methods are repeated only once, which may be about three months, six months, twelve months, eighteen months, two years, three years, four years, five years, or more from the initial assessment.

The invention also features systems for diagnosing the risk for a patient to develop lung cancer, including lung cancer caused by exposure to tobacco smoke. In general, the systems comprise a data structure comprising one or more reference concentrations for an estrogen metabolite and/or an estrogen hormone. The reference concentrations may be concentrations that indicate the subject is healthy, concentrations that indicate the subject is at risk for developing lung cancer (including, for example, a negligible risk, a low risk, a moderately low risk, a moderate risk, a moderately high risk, a high risk, or a severe risk), or concentrations that indicate the subject has lung cancer, and a processor operably connected to the data structure. The processor is preferably capable of comparing, and preferably programmed to compare determined estrogen metabolite and/or estrogen concentrations with reference estrogen metabolite and/or estrogen concentrations. The processor is preferably capable of generating a lung cancer development risk assessment based on the comparison of determined concentrations with reference concentrations. The processor is preferably capable of recommending a treatment regimen that may treat any lung cancer or precancerous state in the subject, or that may delay or prevent the onset of lung cancer in the subject based on the generated risk assessment.

The systems may further comprise a second data structure comprising one or more reference nucleic acid sequences having one or more alterations in the wild type CYP1B1 sequence associated with a probability of surviving lung cancer caused tobacco smoke exposure, and a processor operably connected to the second data structure. Optionally, the second data structure may comprise one or more wild type reference nucleic acid sequences, which have a wild type CYP1B1-encoding nucleic acid sequence. The processor is preferably capable of comparing, and preferably programmed to compare determined nucleic acid sequences (for example, those determined from nucleic acids obtained from a subject) with reference nucleic acid sequences, including wild type reference nucleic acid sequences. The reference nucleic acid sequences, and alterations may comprise any such sequences and alterations described or exemplified herein. Optionally, the processor is capable of determining the sequence of a nucleic acid encoding the CYP1B1 protein in a tissue sample obtained from a subject, including a subject who smokes or had smoked tobacco products. Optionally, the system may comprise an input for accepting determined nucleic acid sequences obtained from tissue samples from a subject. Optionally, the system may comprise an output for providing results of a sequence comparison to a user such as the subject, or a technician, or a medical practitioner. Optionally, the system may comprise a sequencer for determining the sequence of a nucleic acid such as a nucleic acid obtained from a subject. Optionally, the system may comprise a detector for detecting a detectable label on a nucleic acid. Optionally, the system may comprise executable code for causing a programmable processor to determine a prognosis of a lung cancer subject from a comparison of the nucleic acid sequence obtained from a subject to the reference nucleic acid sequence.

The invention also provides computer-readable media comprising executable code for causing a programmable processor to compare estrogen metabolite concentrations and/or estrogen hormone concentration determined from a tissue sample obtained from a subject with one or more reference estrogen metabolite concentrations and/or estrogen hormone concentrations. The computer readable media may further comprise executable code for causing a programmable processor to generate a lung cancer development risk assessment based on the comparison of determined concentrations with reference concentrations. The reference concentrations may be concentrations that indicate the subject is healthy, concentrations that indicate the subject is at risk for developing lung cancer (including, for example, a negligible risk, a low risk, a moderately low risk, a moderate risk, a moderately high risk, a high risk, or a severe risk), or concentrations that indicate the subject has lung cancer. The computer readable media may further comprise executable code for causing a programmable processor to recommend a treatment regimen that may treat any lung cancer or precancerous state in the subject, or that may delay or prevent the onset of lung cancer in the subject based on the generated risk assessment. The computer-readable media may further comprise executable code for causing a programmable processor to compare nucleic acid sequence encoding the CYP1B1 protein determined from a nucleic acid obtained from a tissue sample obtained from a subject with one or more reference nucleic acid sequences having one or more alterations in the wild type nucleic acid sequence encoding the CYP1B1 protein associated with a probability of surviving lung cancer, including lung cancer caused tobacco smoke exposure. Optionally, the computer-readable media may further comprise executable code for causing a programmable processor to compare the nucleic acid sequence of CYP1B1 determined from a nucleic acid obtained from a tissue sample obtained from a subject with one or more wild type reference nucleic acid sequences having a wild type CYP1B1 sequence.

Optionally, the computer-readable media further comprises a processor. In some aspects, computer-readable media may comprise executable code for causing a programmable processor to determine the prognosis of a subject having lung cancer. The computer readable media may comprise executable code for causing a programmable processor to compare a nucleic acid sequence encoding the CYP1B1 protein determined from a polynucleotide obtained from a tissue sample obtained from a subject with one or more reference nucleic acid sequences which have one or more alterations in the wild type CYP1B1 nucleic acid sequence associated with a probability of surviving lung cancer, including lung cancer caused by tobacco smoke exposure.

The computer-readable media may comprise executable code for causing a programmable processor to determine a diagnosis of a subject, for example whether the subject has a risk of developing lung cancer, including lung cancer caused by tobacco smoke exposure. The diagnosis may be based on the comparison of determined nucleic acid sequences with reference nucleic acid sequences. The determined nucleic acids encode the CYP1B1 protein and are compared to the reference nucleic acid sequences, which have one or more alterations in the wild type CYP1B1 nucleic acid sequence associated with a risk of developing lung cancer, including lung cancer from tobacco smoke exposure. Thus, the computer-readable media may comprise an output for providing a diagnosis to a user such as the subject, or a technician, or a medical practitioner.

Computer-readable media may comprise executable code for causing a programmable processor to determine the prognosis of a subject having lung cancer. The computer readable media may comprise executable code for causing a programmable processor to compare estrogen metabolite concentrations and/or estrogen hormone concentrations obtained from a tissue sample obtained from a subject with one or more reference concentrations. The computer-readable media may comprise executable code for causing a programmable processor to determine a diagnosis of a subject, for example whether the subject is healthy, has a risk of developing lung cancer (including, for example, a negligible risk, a low risk, a moderately low risk, a moderate risk, a moderately high risk, a high risk, or a severe risk), or has lung cancer. The diagnosis may be based on the comparison of determined estrogen metabolite concentrations and/or estrogen hormone concentrations. The computer-readable media may comprise an output for providing a diagnosis to a user such as the subject, or a technician, or a medical practitioner.

Estrogen hormones and their respective metabolites may be at elevated concentrations in the lung tissue of a subject because the subject smokes tobacco products. Estrogen hormones and their respective metabolites may also be at elevated concentrations in the lung tissue of a subject because the subject is administered estrogen hormones, for example, as part of a hormone replacement therapy, because the subject is pregnant, or because the subject is administered estrogen-based contraceptives. In addition, it is believed that estrogen synthesis enzymes (e.g., aromatase) and precursors (e.g., testosterone) may be elevated such that higher levels of estrogen metabolites could be localized to where higher concentrations of the enzymes and/or precursors are present.

As with the methods, the one or more estrogen metabolites for use in connection with the systems and computer readable media may comprise one or more of 2-OHE1, 4-OHE1, 4-OHE2, 16-alpha-OHE1, 2-OMeE1, 2-OMeE2 and 2-OHE1, 2-OHE2, 2-hydroxyestrone-3-methyl ether, 4-OMeE1, 4-OMeE2, 17-epiestriol, 16-ketoestradiol, and/or 16-epiestriol. (Fuhrman B J et al. (2012) J. Natl. Cancer Inst. 104:1-14, and, Eliassen A H et al. (2012) Cancer Res. 72(3):696-706). The estrogen may comprise estrone (E₁), estradiol (E₂), and/or estriol (E₃). The metabolites may be produced by the biologic activity of CYP1B1 or a variant thereof such as a variant encoded by a CYP1B1 gene polymorphism, including those described or exemplified herein.

The invention also features prognostic methods, including methods for determining the prognosis of a subject diagnosed with lung cancer, preferably lung cancer caused by tobacco smoke exposure, although not restricted to lung cancer caused by tobacco smoke exposure. These prognostic methods may be used in conjunction with the estrogen metabolite diagnostic methods described above, for example, to assess potential prognoses once a patient at risk for developing lung cancer goes on to develop lung cancer, or these prognostic methods may stand alone, for example, without also evaluating estrogen and estrogen metabolite levels for diagnostic purposes. In the former case (in conjunction with diagnostic methods), such diagnostic methods may further comprise steps for determining the prognosis of a subject diagnosed with lung cancer, including those described below.

Prognostic methods (including those used in conjunction with estrogen and estrogen metabolite diagnostic steps) generally comprise the steps of comparing the sequence of a nucleic acid encoding the CYP1B1 protein obtained from a tissue sample obtained from a subject with one or more reference nucleic acid sequences comprising one or more alterations in the wild type CYP1B1 sequence that are associated with a probability of surviving lung cancer, for example, lung cancer caused by tobacco smoke exposure, determining whether the CYP1B1 nucleic acid sequence obtained from the subject has the alteration based on the comparison and/or determining the subject's prognosis based on the comparison. The comparison may be carried out using a processor programmed to compare nucleic acid sequences, for example, to compare the nucleic acid sequences obtained from the subject with the reference nucleic acid sequences. The methods may optionally include the step of determining the sequence of the nucleic acid encoding the CYP1B1 protein obtained from the subject. A sequence may be determined using deep sequencing methods.

In some aspects, the methods comprise comparing the sequence of a nucleic acid encoding the CYP1B1 protein obtained from a tissue sample obtained from a subject with one or more reference nucleic acid sequences comprising one or more alterations in the wild type CYP1B1 sequence that are associated with a risk of developing lung cancer, determining whether the CYP1B1 nucleic acid sequence obtained from the subject has the alteration based on the comparison and/or diagnosing whether the subject has a risk of developing lung cancer, including lung cancer from tobacco smoke exposure, based on the comparison. The comparing step may be carried out using a processor programmed to compare nucleic acid sequences, for example, to compare the nucleic acid sequences obtained from the subject and the reference nucleic acid sequences. The methods may optionally include the step of determining the sequence of the nucleic acid encoding the CYP1B1 protein obtained from the subject.

From the subject, the tissue sample may be from any tissue in which the nucleic acid sequence encoding the CYP1B1 protein sequence may be obtained. Non-limiting examples of tissues from which a sample may be obtained include blood and lung tissue. The tissue may be a fresh isolate, or may be frozen, or may be fixed, including a formalin-fixed tissue. The methods may include the step of obtaining the tissue sample, and may include the step of obtaining the nucleic acid. The nucleic acid may be any nucleic acid that has, or from which may be obtained, the nucleic acid sequence encoding the CYP1B1 protein, or the complement thereof, or any portion thereof. For example, the nucleic acid may be chromosomal or genomic DNA, may be mRNA, or may be a cDNA obtained from the mRNA. The sequence of the nucleic acid may be determined using any sequencing method suitable in the art.

In some aspects, the methods include hybridization assays. For example, in some detailed aspects, the methods generally comprise contacting a nucleic acid encoding the CYP1B1 protein obtained from a tissue sample obtained from a subject with one or more polynucleotide probes that have a nucleic acid sequence complementary to a CYP1B1 nucleic acid sequence having one or more alterations associated with a probability of surviving lung cancer, and optionally, also with one or more reference probes having a nucleic acid sequence complementary to a wild type CYP1B1 nucleic acid sequence, determining whether the one or more probes, including the one or more reference probes, if used, have hybridized with the nucleic acid encoding the CYP1B1 protein obtained from the subject, and, determining the subject's prognosis based on the determination of whether the probes have hybridized with the nucleic acid obtained from the subject. It is preferred that the one or more polynucleotide probes hybridize under stringent conditions to the CYP1B1 nucleic acid sequence. In some aspects, the methods may further comprise identifying which of the probes hybridized with the nucleic acid, if more than one probe was contacted with the nucleic acid.

In some aspects, the methods for diagnosing a risk of developing lung cancer, including a risk of developing lung cancer from exposure to tobacco smoke, include hybridization assays. For example, in some detailed aspects, the methods generally comprise contacting a nucleic acid encoding the CYP1B1 protein obtained from a tissue sample obtained from a subject with one or more polynucleotide probes that have a nucleic acid sequence complementary to a CYP1B1 nucleic acid sequence having one or more alterations associated with a risk of developing lung cancer, and optionally, also with one or more reference probes having a nucleic acid sequence complementary to a wild type CYP1B1 nucleic acid sequence, determining whether the one or more probes, including the one or more reference probes, if used, have hybridized with the nucleic acid encoding the CYP1B1 protein obtained from the subject, and, determining whether the subject has a risk of developing lung cancer, for example, from tobacco smoke exposure based on the determination of whether the probes have hybridized with the nucleic acid obtained from the subject. It is preferred that the one or more polynucleotide probes hybridize under stringent conditions to the CYP1B1 nucleic acid sequence. In some aspects, the methods may further comprise identifying which of the probes hybridized with the nucleic acid, if more than one probe was contacted with the nucleic acid.

A hybridization assay may be carried out in vitro, and may be carried out using a support such as an array. For example, a nucleic acid obtained from a subject may be labeled and contacted with an array of probes affixed to a support. The probes may comprise DNA or RNA, and may comprise a detectable label.

The one or more polynucleotide probes may comprise a detectable label. The nucleic acid obtained from a subject may be labeled with a detectable label. Thus, in some aspects, the methods may include the step of labeling the nucleic acid obtained from the subject with a detectable label.

Detectable labels may be any suitable chemical label, metal label, enzyme label, fluorescent label, radiolabel, or combination thereof. The methods may comprise detecting the detectable label on probes hybridized with the nucleic acid encoding the CYP1B1 protein. The probes may be affixed to a support, such as an array. For example, a labeled nucleic acid obtained from a subject may be contacted with an array of probes affixed to a support. The probes may include any probes described or exemplified herein.

In another detailed aspect, the hybridization may be carried out in situ, for example, in a cell obtained from the subject. For example, determining the one or more alterations may comprise contacting the cell, or contacting a nucleic acid in the cell, with one or more polynucleotide probes comprising a nucleic acid sequence complementary to a nucleic acid sequence encoding the CYP1B1 protein having one or more alterations associated with a probability of surviving lung cancer, including lung cancer caused by tobacco smoke exposure, and/or associated with a risk of developing lung cancer, including lung cancer caused by tobacco smoke exposure, and comprising a detectable label, and detecting the detectable label on probes hybridized with the nucleic acid encoding the CYP1B1 protein. Detectable labels may be any suitable chemical label, metal label, enzyme label, fluorescent label, radiolabel, or combination thereof.

In any of the hybridization assays, the probes may be DNA or RNA, are preferably single-stranded, and may have any length suitable for avoiding cross-hybridization of the probe with a second target having a similar sequence with the desired target. Suitable lengths are recognized in the art as from about 20 to about 60 nucleotides; optimal for many hybridization assays (for example, see the Resequencing Array Design Guide available from Affymetrix: http://www.affymetrix.com/support/technical/byproduct.affx?product=cseq), though any suitable length may be used, including shorter than 20 or longer than 60 nucleotides, including about 25, about 27, about 30, about 33, about 35, about 37, about 40, about 43, about 45, about 47, about 50, about 53, about 55, or about 57 nucleotides. It is preferred that the probes hybridize under stringent conditions to the CYP1B1 nucleic acid sequence of interest. It is preferred that the probes have 100% complementary identity with the target sequence.

The methods described herein, including the hybridization assays, whether carried out in vitro, on an array, or in situ, may be used to determine any alteration in the nucleic acid sequence encoding the CYP1B1 protein that has a known or suspected association with a probability of surviving lung cancer caused by tobacco smoke exposure and/or with a risk of developing lung cancer, including lung cancer from tobacco smoke exposure, including any of those described or exemplified herein. In any of the methods described herein, the alterations may be, for example, a polymorphism in the CYP1B1-encoding nucleic acid sequence. The polymorphism may comprise one or more nucleotide substitutions, an addition of one or more nucleotides in one or more locations, a deletion of one or more nucleotides in one or more locations, an inversion or other DNA rearrangement, or any combination thereof. A substitution may, but need not, change the amino acid sequence of the CYP1B1 protein. Any number of substitutions, additions, or deletions of nucleotides are possible.

In any of the methods, including methods comprising sequence comparison and methods comprising nucleic acid hybridization, the one or more alterations associated with a probability of surviving lung cancer, including lung cancer caused by tobacco smoke exposure, and/or associated with a risk of developing lung cancer, including lung cancer caused by tobacco smoke exposure, comprises one or more polymorphisms in the gene encoding the CYP1B1 protein. The one or more polymorphisms may indicate whether a subject has an increased risk of developing lung cancer caused by the biologic activity of CYP1B1 (including activity caused by tobacco smoke exposure), or may indicate whether a subject does not have an increased risk of developing lung cancer caused by the biologic activity of CYP1B1 (including activity caused by tobacco smoke exposure). The one or more polymorphisms may indicate whether a subject has a probability of surviving lung cancer caused by the biologic activity of CYP1B1, including a prognosis.

A polymorphism in the gene encoding the CYP1B1 protein may occur at the position corresponding to codon 48 of CYP1B1 cDNA (a CYP1B1 cDNA sequence is provided as SEQ ID NO:4). A polymorphism in the gene encoding the CYP1B1 protein may occur at the position corresponding to codon 119 of CYP1B1 cDNA. A polymorphism in the gene encoding the CYP1B1 protein may occur at the position corresponding to codon 48 and at the position corresponding to codon 119 of CYP1B1 cDNA. A polymorphism in the gene encoding the CYP1B1 protein may occur at the position corresponding to codon 432 of CYP1B1 cDNA. A polymorphism in the gene encoding the CYP1B1 protein may occur at the position corresponding to codon 453 of CYP1B1 cDNA.

A polymorphism at the position corresponding to codon 48 of CYP1B1 cDNA may encode a glycine residue at position 48 in the CYP1B1 protein (SEQ ID NO:10 and SEQ ID NO:11). A polymorphism at the position corresponding to codon 119 of CYP1B1 cDNA may encode a serine residue at position 119 in the CYP1B1 protein (SEQ ID NO:11). A polymorphism at the position corresponding to codon 432 of CYP1B1 cDNA may encode a valine residue at position 432 in the CYP1B1 protein (SEQ ID NO:12). A polymorphism at the position corresponding to codon 453 of CYP1B1 cDNA may encode a serine residue at position 453 in the CYP1B1 protein (SEQ ID NO:13).

A polymorphism at the position corresponding to codon 48 of CYP1B1 cDNA may comprise a change from the codon CGG to the codon GGG at this position (SEQ ID NO:5 and SEQ ID NO:6). A polymorphism at the position corresponding to codon 119 of CYP1B1 cDNA may comprise a change from the codon GCC to the codon TCC at this position (SEQ ID NO:6). A polymorphism at the position corresponding to codon 432 of CYP1B1 cDNA may comprise a change from the codon CTG to the codon GTG at this position (SEQ ID NO:7). A polymorphism at the position corresponding to codon 453 of CYP1B1 cDNA may comprise a change from the codon AAC to the codon AGC at this position (SEQ ID NO:8).

In any of the methods, including methods comprising sequence comparison and methods comprising nucleic acid hybridization, the methods may further comprise determining whether the subject is homozygous or heterozygous for the polymorphism in the gene encoding the CYP1B1 protein. For example, the methods may further comprise determining whether genomic DNA encoding the CYP1B1 protein obtained from the subject is homozygous for the codon GGG at the position corresponding to codon 48 of CYP1B1 cDNA, if it is determined that the nucleic acid encoding the CYP1B1 protein in the tissue sample has the codon GGG at the position corresponding to codon 48 of CYP1B1 cDNA.

A prognosis may relate to, or be measured according to any time frame. For example, the prognosis may comprise a substantial likelihood of mortality within about five years. The prognosis may comprise a substantial likelihood of mortality within about four years. The prognosis may comprise a substantial likelihood of mortality within about three years. The prognosis may comprise a substantial likelihood of mortality within about two years. The prognosis may comprise a substantial likelihood of mortality within about one year. The prognosis may be longer than five years, for example, the prognosis may comprise a substantial likelihood of mortality within about ten years. The prognosis may comprise a substantial likelihood of mortality within about twelve years. The prognosis may comprise a substantial likelihood of mortality within about fifteen years. In some aspects, the prognosis may comprise an about two to about five year range of time. The prognosis may comprise an about three to about five year range of time. The prognosis may comprise an about three to about ten year range of time. The prognosis may comprise an about five to about ten year range of time. Time frames may be shorter than one year or may be longer than five years. Time frames may vary according to clinical standards, or according to the needs or requests from the patient or practitioner.

The inventive methods, whether based on sequence comparison or probe hybridization, may further comprise the steps of treating the subject with a regimen capable of inhibiting the onset of lung cancer. The inventive methods, whether based on sequence comparison or probe hybridization, may further comprise the steps of treating the subject with a regimen capable of improving the prognosis of a lung cancer patient.

The regimen may be tailored to the specific characteristics of the subject, for example, the age, sex, or weight of the subject, the type or stage of the cancer, and the overall health of the subject. In some aspects, the treatment regimen comprises administering to the subject an effective amount of a compound, composition, or biomolecule that inhibits the expression and/or the biologic activity of the CYP1B1 protein. Alternatively, the treatment regimen may comprise inhibiting the expression of the CYP1B1 gene. In some aspects, the treatment regimen comprises one or more of diet management, vitamin supplementation, nutritional supplementation, exercise, psychological counseling, social counseling, education, and regimen compliance management. In some aspects, the treatment regimen comprises preventing, reducing, or eliminating exposure of the subject to tobacco smoke.

The steps of the methods, including any optional steps, may be repeated after a period of time, for example, as a way to monitor a subject's health and prognosis. Repeating the methods may be used, for example, to determine if the patient's prognosis has improved based on a particular treatment regimen, or to determine if adjustments to the treatment regimen should be made to achieve improvement or to attain further improvement in the patient's prognosis. The methods may be repeated at least one time, two times, three times, four times, or five or more times. The methods may be repeated as often as the patient desires, or is willing or able to participate.

The period of time between repeats may vary, and may be regular or irregular. In some aspects, the methods are repeated in three month intervals. In some aspects, the methods are repeated in six month intervals. In some aspects, the methods are repeated in one year intervals. In some aspects, the methods are repeated in two year intervals. In some aspects, the methods are repeated in five year intervals. In some aspects, the methods are repeated only once, which may be about three months, six months, twelve months, eighteen months, two years, three years, four years, five years, or more from the initial assessment.

A subject may be any animal, including mammals such as companion animals, laboratory animals, and non-human primates. Human beings are preferred. In some preferred aspects, the subject is a female human being. In some preferred aspects, the subject is a male human being.

In some aspects, the subject is a non-smoker. In some aspects, the subject periodically smokes tobacco products, or has smoked tobacco products, or may smoke tobacco products. The subject may be (or have been) a light smoker. The subject may be (or have been) a moderate smoker. The subject may be (or have been) a heavy smoker. Tobacco products include, but are not limited to, cigarettes, cigars, pipes, hookahs, and other forms in which tobacco leaves are burned and the resultant smoke inhaled. A Pack Year, typically calculated as the equivalent of a pack of cigarettes (20 cigarettes) per day for a year (including two packs of cigarettes per day for a half year, etc.) may be used as a measurement for the level of tobacco smoking in the subject.

The invention also features a support comprising a plurality of polynucleotide molecules comprising a nucleic acid sequence, or portion thereof, encoding the CYP1B1 protein or portion thereof, and having one or more alterations associated with a probability of surviving lung cancer, including lung cancer caused by tobacco smoke exposure, and/or associated with a risk of developing lung cancer, including lung cancer caused by tobacco smoke exposure, and optionally, a plurality of polynucleotides comprising a nucleic acid sequence, or portion thereof, encoding the wild type CYP1B1 protein or portion thereof. The support may comprise a solid support, and may comprise an array. The polynucleotides may be complementary to the nucleic acid sequence, or portion thereof, encoding the CYP1B1 protein or portion thereof, and having one or more alterations associated with a probability of surviving lung cancer, including lung cancer caused by tobacco smoke exposure, and/or associated with a risk of developing lung cancer, including lung cancer caused by tobacco smoke exposure. The polynucleotides may be complementary to the nucleic acid sequence, or portion thereof, encoding the wild type CYP1B1 protein or portion thereof.

The polynucleotide molecules of the support or array are preferably probes, are preferably complementary to the nucleic acid sequence of interest, and preferably hybridize to the CYP1B1 nucleic acid sequence of interest under stringent conditions. The probes may be DNA or RNA, are preferably single-stranded, and may have any length suitable for avoiding cross-hybridization of the probe with a second target having a similar sequence with the desired target. Suitable lengths may be about 20 to about 60 nucleotides, including about 25, about 30, about 35, about 40, about 45, about 50, or about 55 nucleotides in length. It is preferred that the probes have 100% complementary identity with the target sequence.

The polynucleotide molecules preferably comprise one or more alterations in the nucleic acid sequence of the wild type CYP1B1 gene that are associated with a probability of surviving lung cancer and/or associated with a risk of developing lung cancer. Such alterations include one or more polymorphisms in the gene encoding the CYP1B1 protein.

A polymorphism in the gene encoding the CYP1B1 protein may occur at the position corresponding to codon 48 of CYP1B1 cDNA (a CYP1B1 cDNA sequence is provided as SEQ ID NO:4). A polymorphism in the gene encoding the CYP1B1 protein may occur at the position corresponding to codon 119 of CYP1B1 cDNA. A polymorphism in the gene encoding the CYP1B1 protein may occur at the position corresponding to codon 48 and at the position corresponding to codon 119 of CYP1B1 cDNA. A polymorphism in the gene encoding the CYP1B1 protein may occur at the position corresponding to codon 432 of CYP1B1 cDNA. A polymorphism in the gene encoding the CYP1B1 protein may occur at the position corresponding to codon 453 of CYP1B1 cDNA.

A polymorphism at the position corresponding to codon 48 of CYP1B1 cDNA may encode a glycine residue at position 48 in the CYP1B1 protein (SEQ ID NO:10 and SEQ ID NO:11). A polymorphism at the position corresponding to codon 119 of CYP1B1 cDNA may encode a serine residue at position 119 in the CYP1B1 protein (SEQ ID NO:11). A polymorphism at the position corresponding to codon 432 of CYP1B1 cDNA may encode a valine residue at position 432 in the CYP1B1 protein (SEQ ID NO:12). A polymorphism at the position corresponding to codon 453 of CYP1B1 cDNA may encode a serine residue at position 453 in the CYP1B1 protein (SEQ ID NO:13).

A polymorphism at the position corresponding to codon 48 of CYP1B1 cDNA may comprise a change from the codon CGG to the codon GGG at this position (SEQ ID NO:5 and SEQ ID NO:6). A polymorphism at the position corresponding to codon 119 of CYP1B1 cDNA may comprise a change from the codon GCC to the codon TCC at this position (SEQ ID NO:6). A polymorphism at the position corresponding to codon 432 of CYP1B1 cDNA may comprise a change from the codon CTG to the codon GTG at this position (SEQ ID NO:7). A polymorphism at the position corresponding to codon 453 of CYP1B1 cDNA may comprise a change from the codon AAC to the codon AGC at this position (SEQ ID NO:8).

The invention also features systems for determining the prognosis of a patient having lung cancer caused by exposure to tobacco smoke. In general, the systems comprise a data structure comprising one or more reference nucleic acid sequences having one or more alterations in the wild type CYP1B1 sequence associated with a probability of surviving lung cancer caused tobacco smoke exposure, and a processor operably connected to the data structure. Optionally, the data structure may comprise one or more wild type reference nucleic acid sequences, which have a wild type CYP1B1-encoding nucleic acid sequence. The processor is preferably capable of comparing, and preferably programmed to compare determined nucleic acid sequences (for example, those determined from nucleic acids obtained from a subject) with reference nucleic acid sequences, including wild type reference nucleic acid sequences.

The reference nucleic acid sequences may comprise the one or more alterations described or exemplified herein. The alterations may comprise, for example, a polymorphism in the gene encoding the CYP1B1 protein may occur at the position corresponding to codon 48 of CYP1B1 cDNA (CYP1B1 cDNA presented in SEQ ID NO:4). A polymorphism in the gene encoding the CYP1B1 protein may occur at the position corresponding to codon 119 of CYP1B1 cDNA. A polymorphism in the gene encoding the CYP1B1 protein may occur at the position corresponding to codon 48 and at the position corresponding to codon 119 of CYP1B1 cDNA. A polymorphism in the gene encoding the CYP1B1 protein may occur at the position corresponding to codon 432 of CYP1B1 cDNA. A polymorphism in the gene encoding the CYP1B1 protein may occur at the position corresponding to codon 453 of CYP1B1 cDNA.

A polymorphism at the position corresponding to codon 48 of CYP1B1 cDNA may encode a glycine residue at position 48 in the CYP1B1 protein (SEQ ID NO:10 and SEQ ID NO:11). A polymorphism at the position corresponding to codon 119 of CYP1B1 cDNA may encode a serine residue at position 119 in the CYP1B1 protein (SEQ ID NO:11). A polymorphism at the position corresponding to codon 432 of CYP1B1 cDNA may encode a valine residue at position 432 in the CYP1B1 protein (SEQ ID NO:12). A polymorphism at the position corresponding to codon 453 of CYP1B1 cDNA may encode a serine residue at position 453 in the CYP1B1 protein (SEQ ID NO:13).

A polymorphism at the position corresponding to codon 48 of CYP1B1 cDNA may comprise a change from the codon CGG to the codon GGG at this position (SEQ ID NO:5 and SEQ ID NO:6). A polymorphism at the position corresponding to codon 119 of CYP1B1 cDNA may comprise a change from the codon GCC to the codon TCC at this position (SEQ ID NO:6). A polymorphism at the position corresponding to codon 432 of CYP1B1 cDNA may comprise a change from the codon CTG to the codon GTG at this position (SEQ ID NO:7). A polymorphism at the position corresponding to codon 453 of CYP1B1 cDNA may comprise a change from the codon AAC to the codon AGC at this position (SEQ ID NO:8).

Optionally, the processor is capable of determining the sequence of a nucleic acid encoding the CYP1B1 protein in a tissue sample obtained from a subject, including a subject who smokes or had smoked tobacco products. Optionally, the system may comprise an input for accepting determined nucleic acid sequences obtained from tissue samples from a subject. Optionally, the system may comprise an output for providing results of a sequence comparison to a user such as the subject, or a technician, or a medical practitioner. Optionally, the system may comprise a sequencer for determining the sequence of a nucleic acid such as a nucleic acid obtained from a subject. Optionally, the system may comprise a detector for detecting a detectable label on a nucleic acid. Optionally, the system may comprise executable code for causing a programmable processor to determine a prognosis of a lung cancer subject from a comparison of the nucleic acid sequence obtained from a subject to the reference nucleic acid sequence.

In any of the systems, a computer may comprise the processor or processors used for determining information, comparing information and determining results. The computer may comprise computer-readable media comprising executable code for causing a programmable processor to determine a diagnosis of the subject. The systems may comprise a computer network connection, including an Internet connection.

The invention also provides computer-readable media. In general, the computer-readable media comprise executable code for causing a programmable processor to compare nucleic acid sequence encoding the CYP1B1 protein determined from a nucleic acid obtained from a tissue sample obtained from a subject with one or more reference nucleic acid sequences having one or more alterations in the wild type nucleic acid sequence encoding the CYP1B1 protein associated with a probability of surviving lung cancer, including lung cancer caused tobacco smoke exposure. Optionally, the computer-readable media comprise executable code for causing a programmable processor to compare the nucleic acid sequence of CYP1B1 determined from a nucleic acid obtained from a tissue sample obtained from a subject with one or more wild type reference nucleic acid sequences having a wild type CYP1B1 sequence.

Optionally, the computer-readable media further comprises a processor. Computer-readable media may comprise executable code for causing a programmable processor to determine the prognosis of a subject having lung cancer. The computer readable media may comprise executable code for causing a programmable processor to compare a nucleic acid sequence encoding the CYP1B1 protein determined from a polynucleotide obtained from a tissue sample obtained from a subject with one or more reference nucleic acid sequences which have one or more alterations in the wild type CYP1B1 nucleic acid sequence associated with a probability of surviving lung cancer, including lung cancer caused by tobacco smoke exposure.

The computer-readable media may comprise executable code for causing a programmable processor to determine a diagnosis of a subject, for example whether the subject has a risk of developing lung cancer, including lung cancer caused by tobacco smoke exposure. The diagnosis may be based on the comparison of determined nucleic acid sequences with reference nucleic acid sequences. The determined nucleic acids encode the CYP1B1 protein and are compared to the reference nucleic acid sequences, which have one or more alterations in the wild type CYP1B1 nucleic acid sequence associated with a risk of developing lung cancer, including lung cancer from tobacco smoke exposure. Thus, the computer-readable media may comprise an output for providing a diagnosis to a user such as the subject, or a technician, or a medical practitioner.

The reference nucleic acid sequences may comprise any of the one or more alterations described or exemplified herein. The alterations may be, for example, a polymorphism in the gene encoding the CYP1B1 protein may occur at the position corresponding to codon 48 of CYP1B1 cDNA (a CYP1B1 cDNA sequence is provided as SEQ ID NO:4). A polymorphism in the gene encoding the CYP1B1 protein may occur at the position corresponding to codon 119 of CYP1B1 cDNA. A polymorphism in the gene encoding the CYP1B1 protein may occur at the position corresponding to codon 48 and at the position corresponding to codon 119 of CYP1B1 cDNA. A polymorphism in the gene encoding the CYP1B1 protein may occur at the position corresponding to codon 432 of CYP1B1 cDNA. A polymorphism in the gene encoding the CYP1B1 protein may occur at the position corresponding to codon 453 of CYP1B1 cDNA.

A polymorphism at the position corresponding to codon 48 of CYP1B1 cDNA may encode a glycine residue at position 48 in the CYP1B1 protein (SEQ ID NO:10 and SEQ ID NO:11). A polymorphism at the position corresponding to codon 119 of CYP1B1 cDNA may encode a serine residue at position 119 in the CYP1B1 protein (SEQ ID NO:11). A polymorphism at the position corresponding to codon 432 of CYP1B1 cDNA may encode a valine residue at position 432 in the CYP1B1 protein (SEQ ID NO:12). A polymorphism at the position corresponding to codon 453 of CYP1B1 cDNA may encode a serine residue at position 453 in the CYP1B1 protein (SEQ ID NO:13).

A polymorphism at the position corresponding to codon 48 of CYP1B1 cDNA may comprise a change from the codon CGG to the codon GGG at this position (SEQ ID NO:5 and SEQ ID NO:6). A polymorphism at the position corresponding to codon 119 of CYP1B1 cDNA may comprise a change from the codon GCC to the codon TCC at this position (SEQ ID NO:6). A polymorphism at the position corresponding to codon 432 of CYP1B1 cDNA may comprise a change from the codon CTG to the codon GTG at this position (SEQ ID NO:7). A polymorphism at the position corresponding to codon 453 of CYP1B1 cDNA may comprise a change from the codon AAC to the codon AGC at this position (SEQ ID NO:8).

The systems and computer-readable media may be used in any of the methods described or exemplified herein, for example, methods for identifying alterations in the CYP1B1 gene, and methods for determining the prognosis of a lung cancer patient, or methods for diagnosing a risk of developing lung cancer, including lung cancer from tobacco smoke exposure. For example, the systems and computer-readable media may be used to facilitate comparisons of gene sequences, or to facilitate determining a prognosis or a diagnosis.

The following examples are provided to describe the invention in greater detail. They are intended to illustrate, not to limit, the invention.

### EXAMPLE 1

### Materials And Methods

Study Population. A total of 220 DNA samples from lung cancer patients and healthy control individuals collected between October 1992 and December 1997 were evaluated. Patients were diagnosed with lung cancer at the Fox Chase Cancer Center (FCCC). Control subjects were employees of FCCC and members of the community. Samples correspond to a subset of a population from which DNA was available for analysis. This study was approved by the Institutional Review Board at FCCC.

Demographic information, including age, gender, race, and smoking status was obtained by questionnaire. Individuals were classified as nonsmokers and smokers (former and current) based on self-reported questionnaire data. Smokers were further categorized as light smokers (pack-years <40) or heavy smokers (pack-years ≥40). The tumor histology, clinical stage (based on the TNM system (Primary Tumor, Regional Lymph Nodes, and Distant Metastasis)), treatment in addition to surgery (which included induction therapy, adjuvant chemotherapy or radiation therapy), and date of death were collected from the patient's medical record. Survival was defined as the time (in months) from initial surgery until the most recent follow-up appointment at FCCC or death. Approximately one third of the patients received some type of additional treatment including induction or adjuvant chemotherapy (10%) or radiation therapy (19%).

DNA Genotyping. Genetic polymorphisms in CYP1B1 (C>G, G>T, C>G and A>G at codons 48, 119, 432 and 453, respectively) and GSTM1 (deletion) were determined using TaqMan® (Roche Molecular Systems, Pleasanton, Calif.) assays (Applied Biosystems, Foster City, Calif.). The PCR reactions for allelic discrimination were performed based on instructions from the manufacturer. Briefly, reactions (25 μL) were prepared in 96-well reaction plates using 1× TaqMan® Universal PCR Master mix without AmpErase® (Roche Molecular Systems, Pleasanton, Calif.) UNG, 1× Taqman SNP Genotyping Assay and 20 ng of genomic DNA. In the case of the GSTM1 gene deletion assay, 1× RNase P assay (Hs02575461_cn) was run in parallel as a two-copy reference gene. The reaction conditions were: 10 minutes at 95° C. followed by 40 cycles of 15 seconds at 95° C. and 1 minute at 60° C. Reactions were performed in the ABI PRISM 7900HT instrument (Applied Biosystems). Allelic discrimination plots were generated using automated detection software (SDS, Applied Biosystems).

The polymorphism in CYP1A1 (A>G at codon 462) was determined by pyrosequencing reaction. Genomic DNA (40 ng) was amplified using 2 units of Platinum® (Invitrogen Corp., Carlsbad, Calif.) Taq DNA Polymerase High Fidelity (Invitrogen, Carlsbad, Calif.), 1× High Fidelity PCR Buffer (Invitrogen), 0.2 μM dNTP mixture and 0.2 μM of each primer (forward: gctgtctccctctggtta (SEQ ID NO:1); reverse: cgttgcagcaggatagcc-biotin labeled) (SEQ ID NO:2) in a final volume of 50 μl. The reaction conditions were: 5 minutes at 95° C. followed by 35 cycles of 30 seconds at 95° C., 30 seconds at 54° C., and 72° C. for 45 seconds plus a final cycle of 5 minutes at 72° C. The biotinylated PCR products (20 μl) were immobilized on Streptavidin-coated Sepharose® (GE Healthcare Biosciences A.B., Sweden) High Performance beads (Amersham Biosciences, Piscataway, N.J.) and processed to obtain a single-stranded DNA using the PSQ™ 96 Sample Preparation Kit and the PSQ™ Vacuum Prep Tool (Biotage, Uppsala, Sweden), according to the manufacturer's instructions. The template was incubated subsequently with 0.4 μM of sequencing primer (gcggaagtgtatcggtga) (SEQ ID NO:3) at 80′C for 2 minutes in a PSQ™ 96-plate (Biotage). The sequencing-by-synthesis reaction of the complementary strand was automatically performed on a PSQ™ 96MA instrument (Biotage) at room temperature using PyroGold reagents (Biotage). Allelic discrimination and quality assessment of the raw data were performed automatically using PSQ™ 96 SNP Software (Biotage).

Genotyping results for CYP1A1 from a previous study conducted more than a decade ago were confirmed using more efficient sequencing strategies. The use of pyrosequencing allowed the identification of an additional polymorphism (C>A at codon 461) adjacent to codon 462 that would have interfered with the restriction enzyme-based method used previously.

Statistical Analysis. Hardy-Weinberg equilibrium of CYP1A1 and CYP1B1 variants was assessed for both cases and controls among Caucasian subjects using Haploview. Haploview was also used to estimate haplotype frequencies based on the standard expectation-maximization algorithm and to calculate the pairwise linkage disequilibrium (LD) among CYP1B1 variants.

The association between the frequency of CYP1B1, CYP1A1 and GSTM1 genotypes and cancer incidence was assessed for each polymorphism individually via Chi-square and Fisher's exact tests and multivariable logistic regression. Demographic factors such as age, gender, pack-years of smoking, as well as their interactions with polymorphic genotypes, were included as covariates in the multivariable model. If the interactions were significant, the data were stratified based on the factor involved (age, pack-years of smoking) and the subset was analyzed accordingly.

The impact of genotypes on overall survival was assessed using the Kaplan-Meier estimation method and the Cox proportional hazards model. Clinical and demographic factors such as age, gender, smoking status, tumor histology, clinical stage, and adjuvant treatment were included as covariates. Survival data were analyzed independently for all cases and Caucasians only.

For the multivariable analyses (cancer risk and patient survival), stepwise variable selection methods were used to identify the most parsimonious models. Because of the large number of tests and the limited number of observations, the data were not corrected for multiple comparisons.

### EXAMPLE 2

### Results

The demographic characteristics of the 220 samples evaluated (N=113 controls and 107 cases) are presented in Table 1, including gender, race, age, smoking history and tumor type. The majority of the samples were Caucasian (>95%), and the frequency of men with cancer (56%) was significantly higher than that of women (p<0.001, Chi-square test). The prevalent tumor types were adenocarcinoma (38.7%) and squamous cell carcinoma (33.0%). Among the controls, the frequency of female heavy smokers (35%) was significantly lower than that of males (56%) (p<0.006, Chi-square test). However, a significantly higher frequency of female heavy smokers (61%) was observed within the cases as compared to the same category in the control group (p<0.003, Chi-square test).

TABLE 1
Demographics and Smoking History
of Controls and Cancer Patients

| | Controls | Cancer patients^{a} |
|---|---|---|
| Sample size | 113 | 107 |
| Sex (%) | | |
| Men | 31 | 56^{b} |
| Race (%) | | |
| Caucasian | 97 | 95 |
| African-American | 3 | 4 |
| Asian | 0 | 1 |
| Age (years) | | |
| Range | 45-88 | 34-88 |
| Mean ± SD | | |
| Men | 59 ± 11.2 | 67 ± 10.3^{c} |
| Women | 62 ± 12.2 | 63 ± 11.7 |
| Smoking history | | |
| Smokers (%) | | |
| Men | 97^{b} | 100^{b} |
| Women | 73 | 81 |
| Heavy Smokers (%) | | |
| (pack-year ≥ 40) | | |
| Men | 56 | 64 |
| Women | 35^{d} | 61^{c} |

^{a}Tumors were adenocarcinoma (38.7%), squamous cell (33.0%), bronchioloalveolar (11.3%), large cell carcinomas (17%). Tumors were at clinical stages I (42.1%), II (26.1%), IIIA (21.5%), IIIB and IV (5.6%) or not available (4.7%).
^{b}Significantly different from women by Chi-square test, p < 0.001.
^{c}Significantly different from controls by Chi-square test, p < 0.003.
^{d}Significantly different from men by Chi-square test, p = 0.006.

Genotype frequencies of CYP1B1 (codons 48, 119, 432 and 453) and CYP1A1 (codon 462) showed no significant deviations from Hardy-Weinberg equilibrium in either cases or controls. However, the four CYP1B1 loci demonstrate significant pairwise LD in both cases and controls. D′ values exceeded 0.85 (95% CI between 0.47 and 1) for all pairwise combinations. Polymorphisms in codons 48 and 119 of the CYP1B1 showed the strongest LD (D′=1; 95% CI [0.95, 1]) in both cases and controls. In all individuals, except in one control and one case, the presence of the G and C alleles at codon 48 was linked to the T and G alleles at codon 119, respectively. Thus, the data for codon 119 were not included in subsequent statistical analyses.

The remaining pairwise combinations suggested a significant but weaker LD as indicated by lower bounds of 95% confidence intervals of 0.47. The linkage observed between polymorphisms at codons 48 and 119 has been previously described.

Of the 16 possible haplotypes for the CYP1B1 gene, four had estimated frequencies of at least 1% and accounted for the majority of the samples (97.2% and 98.9% of controls and cases, respectively) (Table 2). The haplotype GTCA (codons 48, 119, 432 and 453, respectively; CYP1B1*2 allele) was increased significantly in cancer patients compared to controls (X² p value=0.027); however, this observation was no longer significant after performing a permutation test on the haplotypes and adjusting for multiple comparisons.

TABLE 2
Haplotype frequency estimation for CYP1B1 in Caucasians

| Haplotypes (codon) | | | | | | Frequencies^{b} (%) | | Chi-square |
|---|---|---|---|---|---|---|---|---|
| 48 | 119 | 432 | 453 | Amino acid change | Allele^{a} | Cancer | Controls | p value |
| C | G | G | A | L432V | CYP1B1*3 | 36.0 | 43.0 | 0.144 |
| G | T | C | A | R48G; A119S | CYP1B1*2 | 35.4 | 25.5 | 0.027^{c} |
| C | G | C | G | N453S | CYP1B1*4 | 20.4 | 19.4 | 0.788 |
| C | G | C | A | None (Wild-type allele) | CYP1B1*1 | 7.1 | 9.3 | 0.404 |

^{a}Allele denomination recommended by the Human Cytochrome P450 Allele Nomenclature Committee. Two alleles indicated in the table do not have denomination (ND).
^{b}Haplotype frequency estimated by the expectation-maximization algorithm.
^{c}Significantly different between cancer and control cases by Chi-square test.

Lung Cancer Risk. The genotypic frequencies of polymorphisms in the CYP1B1, CYP1A1 and GSTM1 genes in all samples (Table 3) was used for analysis of the effect of polymorphisms on lung cancer risk and patient survival. Logistic regression analyses indicated that both the CYP1B1 polymorphism at codon 432 and deletion of GSTM1 are associated with an increased risk of lung cancer development in smokers. With respect to CYP1B1, homozygous wild-type individuals at codon 432 (CC) who are light smokers (<40 pack-years) were at an approximate 5-fold increased risk of developing lung cancer as compared to heterozygous individuals (GC) (OR 5.5, p=0.005) (Table 4). No significant association between polymorphisms in codon 432 and lung cancer risk was observed when all smokers (heavy and light smokers) were analyzed (data not shown). With respect to GSTM1, smokers with a deletion of this gene (null) had an approximate 2-fold elevated risk of lung cancer (OR 1.84) as compared to nonsmokers, but the trend did not reach statistical significance (p=0.061) (data not shown). This association achieved significance when only heavy smokers (≥40 pack-years) were evaluated (OR 2.8; p=0.025) (Table 4).

TABLE 3
Frequency of Genotypes in Cases and Controls

| | | |
|---|---|---|
| Genotype | Cases N (%)^{a} | Controls N (%)^{a} |

CYP1B1

| | | | | | |
|---|---|---|---|---|---|
| codon 48 | CC | 43 | (40.2) | 62 | (54.9) |
| | GC | 52 | (48.6) | 42 | (37.2) |
| | GG | 12 | (11.2) | 9 | (8.0) |
| codon 119 | GG | 43 | (40.2) | 61 | (54.0) |
| | GT | 51 | (47.7) | 43 | (38.1) |
| | TT | 13 | (12.2) | 9 | (8.0) |
| codon 432 | CC | 42 | (39.3) | 32 | (28.3) |
| | GC | 49 | (45.8) | 57 | (50.4) |
| | GG | 16 | (15.0) | 24 | (21.2) |
| codon 453 | AA | 67 | (62.6) | 74 | (65.5) |
| | GA | 36 | (33.6) | 32 | (28.3) |
| | GG | 4 | (3.7) | 7 | (6.2) |

CYP1A1

| | | | | | |
|---|---|---|---|---|---|
| codon 462 | AA | 101 | (94.4) | 110 | (97.4) |
| | AG | 6 | (5.6) | 3 | (2.7) |

GSTM1

| | | | | |
|---|---|---|---|---|
| WT^{b} | 46 | (43.0) | 53 | (46.9) |
| null | 61 | (57.0) | 60 | (53.1) |

^{a}Values represent the percentage of the total number of individuals possessing a particular genotype.
^{b}Individuals with one or two copies of the GSTM1 gene were categorized as wild-type (WT).

TABLE 4
Multivariable Analysis for Lung Cancer Cases versus
Controls Stratified by Genotype and Smoking History^{a}

| | | |
|---|---|---|
| Odds ratio^{b} | 95% CI | p value |

CYP1B1 codon 432 CC versus GC

| | | | |
|---|---|---|---|
| Light smokers (<40 pack-years) | 5.5 | 1.7-18.0 | 0.005 |
| Heavy smokers (≥40 pack-years) | 0.7 | 0.3-1.9 | 0.523 |

CYP1B1 codon 432 CC versus GG

| | | | |
|---|---|---|---|
| Light smokers (<40 pack-years) | 3.4 | 0.8-14.1 | 0.090 |
| Heavy smokers (≥40 pack-years) | 1.8 | 0.6-5.9 | 0.300 |

GSTM1 null versus WT

| | | | |
|---|---|---|---|
| Light smokers (<40 pack-years) | 1.2 | 0.5-3.2 | 0.650 |
| Heavy smokers (≥40 pack-years) | 2.8 | 1.1-6.7 | 0.030 |

^{a}Only polymorphisms showing statistical significance have been presented.
^{b}Controlling for age and gender.

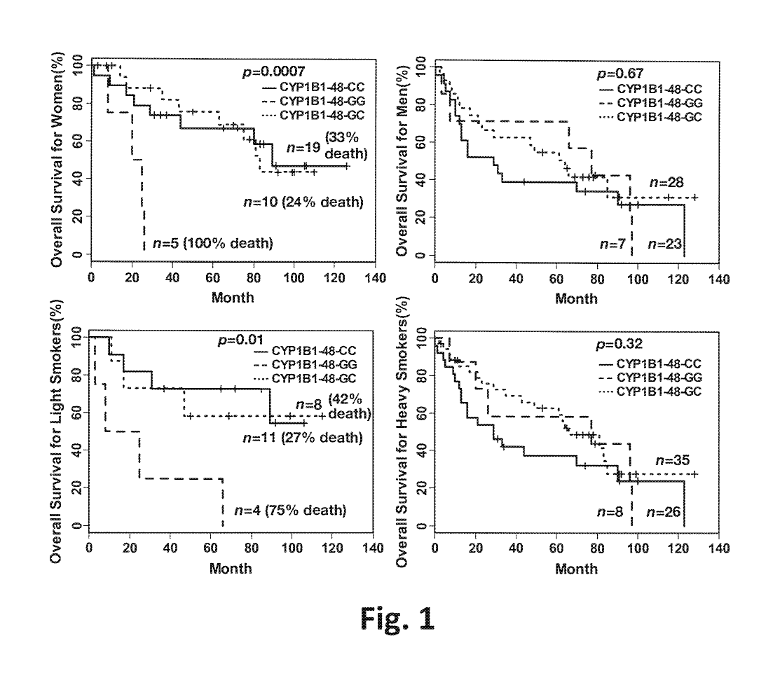

Lung Cancer Survival. The effect of genetic polymorphisms on the overall survival of lung cancer patients was determined in 101 cases with clinical stages I, II and IIIA. Very few patients with clinical stages IIIB and IV (N=6) were present in the data set; thus, these individuals with advanced stage lung cancer were excluded from survival analyses. The median follow-up time after surgery was 47 months (range: 0-128). 58.4% of the patients died prior to the study, with a median follow-up time of 23 months (range: 0 123). As expected, patients still alive at the time of the study (41.6%) exhibited a longer follow-up time (median=75 months, range: 3-128). Univariate analysis (Kaplan-Meier estimation) showed that none of the women carrying the variant genotype GG at codon 48 of the CYP1B1 gene, which confers increased basal CYP1B1 gene expression, were alive after 5 years of follow-up as compared to women carrying the CC or GC genotypes (67% and 76% survival, respectively) (FIG. 1, upper panels). After controlling for covariates (age, smoking status and pack-years of smoking, tumor histology, clinical stage, and adjuvant treatment), the analysis revealed that the survival time of women homozygous for the variant genotype (GG) at codon 48 of the CYP1B1 gene was significantly less than that of women carrying either the CC genotype (hazard ratio (HR) 16.13; p<0.001; 95% CI 4-75) or the GC genotype (HR 45.45; p<0.001; 95% CI 6-329) (Table 5). This association was not significant for men.

TABLE 5
Multivariable Analysis for Survival of Lung Cancer
Cases Stratified by Gender and Smoking History

| CYP1B1 codon 48 | | GG versus GC | | GG versus CC | |
|---|---|---|---|---|---|
| | | Hazard ratio^{a} (p value; 95% CI) | | | |
| Gender | Women | 45.45 | (p = 0.0002; 6-329) | 16.13 | (p = 0.0004; 4-75) |
| | Men | 0.67^{c} | (p = 0.48; 0.22-2.04) | 0.54^{c} | (p = 0.28; 0.78-1.66) |
| | | Hazard ratio^{b} (p value; 95% CI) | | | |
| Smoking | Light smokers | 5.29 | (p = 0.0247; 1.24-22.58) | 7.94 | (p = 0.0045; 1.9-32.9) |
| | (<40 pack-years) | | | | |
| | Heavy smokers | 0.49^{c} | (p = 0.28; 0.14-1.76) | 0.44^{c} | (p = 0.11; 0.16-1.22) |

(≥40 pack-years)

^{a}Controlling for age, smoking status, tumor histology, clinical stage, adjuvant treatment.
^{b}Controlling for age, gender, tumor histology, clinical stage, adjuvant treatment.
^{c}NS = Nonsignificant.

Univariate analysis, stratifying the group of smokers as light (<40 pack-years) or heavy (≥40 pack-years), revealed that the polymorphism at codon 48 of the CYP1B1 gene was significantly associated with the survival of light smokers with lung cancer. The 5-year survival rates and 95% CIs for light smokers carrying the genotypes GG (homozygous variant), GC and CC were 25% (9-67%), 58% (18-84%) and 73% (37-90%) respectively, p=0.01) (FIG. 1, lower panels). Multivariable analyses controlling for covariates (age, gender, tumor histology, clinical stage, and adjuvant treatment) showed that the survival time of light smokers carrying the CYP1B1 variant genotype GG was significantly less than that of light smokers carrying either the CC genotype (HR 7.94; 95% CI 1.9-32.9; p=0.005) or the GC genotype (HR 5.29; 95% CI 1.24-22.58; p=0.02). No significant difference was observed among heavy smokers. Although there were more light smokers (52%) compared to heavy smokers (35%) among women, this difference was not significant (p=0.12). To further confirm that this result was not biased by gender, the effect of the polymorphism at codon 48 of the CYP1B1 gene on survival status was also analyzed in four different subpopulations: female light smokers, female heavy smokers, male light smokers, male heavy smokers. No significant results were obtained from this analysis, which may be due to the small sample size.

Multivariable analyses stratified by pack-years and including only Caucasians, the race of 95% of the cancer patients, were also performed. Unlike the result discussed above (FIG. 1, lower panels), this analysis failed to identify a significant difference (p≥0.05) in survival among light smokers. However, stratification by gender instead of pack-years revealed a significant association of the variant genotype at codon 48 with the shorter survival of women with lung cancer, corroborating the data presented in FIG. 1 (upper panels).

Finally, the combined polymorphisms in CYP1B1 (codons 48, 119, 432 and 453), CYP1A1 (codon 462) and the GSTM1 deletion had no significant effect on either the incidence of lung cancer or patient survival.

### EXAMPLE 3

### Summary

These data represent a study to simultaneously investigate multiple polymorphisms in CYP1B1 (codons 48, 119, 432 and 453), CYP1A1 (codon 462) and GSTM1 deletion with respect to lung cancer risk and to report the effect of a polymorphism at codon 48 of CYP1B1 on the survival of lung cancer patients. The homozygous variant allele (GG) at codon 48, which is completely linked to codon 119, was associated with a dramatic reduction in the survival time of both women and light smokers (men and women) with lung cancer (FIG. 1). One important observation was that all women carrying the GG genotype died within 5 years of surgery (0% survival rate) as compared to more than 77% survival among women carrying either the CC or GC genotype (FIG. 1, upper panels). This observation was the same when all cases or just Caucasians were analyzed. But this genotype was present in only 5 women (10.9%) and 7 (11.7%) men in the study. Without intending to be limited to any particular theory or mechanism of action, one possible explanation is that the shorter survival of women with the homozygous variant genotype at codon 48 (GG) and/or codon 119 (TT) may be a consequence of an alteration in the metabolism of estrogen by the CYP1B1 enzyme.

The homozygous variant genotype at codon 48 (GG) was also significantly associated with shorter survival among light smokers. As mentioned in Example 1, this analysis included all 107 cases with lung cancer and was adjusted for gender and other covariates (age, tumor histology, clinical stage, and adjuvant treatment). However, this association was not observed when a similar analysis was performed with only Caucasians (N=89 or 95% of cases).

A significant association was observed between the CYP1B1 polymorphism at codon 432 or the GSTM1 gene deletion and lung cancer incidence only among smokers (Table 4). Light smokers carrying the homozygous wild-type allele at codon 432 (CC) of the CYP1B1 gene were at an elevated risk of lung cancer as compared to those with the GC genotype (Table 4).

It was also observed that heavy smokers carrying a deletion of the GSTM1 gene were at an approximate 3-fold elevated risk of lung cancer (Table 4). This effect was not observed when all patients (light and heavy smokers and nonsmokers) were considered. Similarly, no association between GSTM1 deletion alone and lung cancer risk was observed previously when a larger data set was analyzed.

### EXAMPLE 4

### Tobacco Smoke Modulates Estrogen Metabolism in the Mouse Lung

Recent studies suggest that the female hormone estrogen promotes lung cancer development. However, the relationship between tobacco smoke exposure and estrogen is not well studied. Previous investigations showed that whole-body exposure to tobacco smoke induced the expression of the phase I detoxification enzyme cytochrome P450 1B1 (CYP1B1) within the lungs of female A/J mice. CYP1B1 activates polyaromatic hydrocarbons in tobacco smoke and also converts estrogen to catechol metabolites, in particular 4-hydroxy estrogens (4-OHEs), which are known to be carcinogenic.

Animals. Heterozygous 129/SvJ Cyp1b1-KO mice were purchased from the Mutant Mouse Regional Resource Center (MMRRC, supported by National Center for Research Resources-National Institutes of Health) and bred to homozygosity in-house, then maintained on Teklad Global 18% Protein Rodent Diet 2018S. Smoke exposures were performed on female C57/B6 mice (1.5-2 years old) carrying a human APOE*4 transgene that were part of an atherosclerosis study at Duke University and fed a high-fat diet TD.88051. Animals had free access to food and water. All animal experimentation was approved by the Institutional Animal Care and Use Committees at Fox Chase Cancer Center and Duke University.

Genotyping. PCR-based genotyping of Cyp1b1 wild-type (Cyp1b1-WT) and knockout (Cyp1b1-KO) mice was performed using Choice Taq Blue DNA Polymerase Master mix (Denville Scientific, South Plainfield, N.J.) according to the following protocol: Wild-type primers—AAATCAAAACAGATACCCGGATG (SEQ ID NO:14) versus TCCGGCCTCTCACTTGCA (SEQ ID NO:15); KO/Neo primers—TGAATGAACTGCAGGACGAG (SEQ ID NO:16) versus ACGACTTGGGCTTAATGGTC (SEQ ID NO:17); reaction conditions—95° C. for 5 min, followed by 35 cycles at 95° C. for 30 s, 60° C. for 1 min and 72° C. for 30 s.

Tobacco smoke exposure. Mainstream and sidestream cigarette smoke was pumped into sealed chambers (via sidestream exposure) containing APOE*4 transgenic mice using a custom-built microprocessor-controlled cigarette-smoking machine (Model TE-10z; Teague Enterprises, Davis, Calif.). This machine provided quantitative volumes of sidestream smoke from eight cigarettes [University of Kentucky reference cigarette (2R4F)] per cycle (8 min). The animals were exposed to smoke for 2 h per day, 5 days per week for 8 weeks. The total suspended particulate was 100-120 mg/m³ and the carbon monoxide (CO) levels were 600-800 p.p.m. Animals remained unrestrained in their cages during smoke exposure, with full access to food and water.

Lung tissue collection. Following euthanasia, the lungs were perfused by intracardiac injection with 30 ml phosphate buffered saline to flush out the blood. Perfused tissues were snap-frozen in liquid nitrogen and stored at −80° C. The accessory lobe of the lung was reserved for RNA extraction, whereas the remaining lobes were processed for estrogen metabolite analyses.

Measurement of estrogen and its metabolites. Reagents and materials. Estrogens and EM, including E₁, E₂, estriol (E₃), 16-epiestriol (16-epiE₃), 17-epiestriol (17-epiE₃), 16-ketoestradiol (16-ketoE₂), 16α-hydroxyestrone (16α-OHE₁), 2-methoxyestrone (2-MeOE₁), 4-methoxyestrone (4-MeOE₁), 2-hydroxyestrone-3-methyl ether (3-MeOE₁), 2-methoxyestradiol (2-MeOE₂), 4-methoxyestradiol (4-MeOE₂), 2-hydroxyestrone (2-OHE₁), 4-hydroxyestrone (4-OHE1), 2-hydroxyestradiol (2-OHE₂) and 4-hydroxyestradiol (4-OHE₂), were obtained from Steraloids (Newport, R.I.). Stable isotope-labeled estrogens (SI-EM), including estradiol-13,14,15,16,17,18-13C₆ (13C₆-E₂) and estrone-13,14,15,16,17,18-13C₆ (13C₆-E₁), were purchased from Cambridge Isotope Laboratories (Andover, Mass.); estriol-2,4,17-d₃ (d₃-E₃), 2-hydroxyestradiol-1,4,16,16,17-d₅ (d₅-2-OHE₂) and 2-methoxyestradiol-1,4,16,16,17-d₅ (d₅-2-MeOE₂) were obtained from C/D/N Isotopes (Pointe-Claire, Quebec, Canada). 16-Epiestriol-2,4,16-d₃ (d₃-16-epiE₃) was purchased from Medical Isotopes (Pelham, N.H.). All steroid analytical standards have reported chemical and isotopic purity_98% and were used without further purification. Dichloromethane and methanol were obtained from EM Science (Gibbstown, N.J.). Glacial acetic acid and sodium bicarbonate were purchased from J. T. Baker (Phillipsburg, N.J.), and sodium hydroxide and sodium acetate were purchased from Fisher Scientific (Fair Lawn, N.J.). Ethyl alcohol was obtained from Pharmco Products (Brookfield, Conn.). Formic acid, acetone, dansyl chloride and I-ascorbic acid were obtained from Sigma-Aldrich Chemical Co. (St Louis, Mo.). All chemicals and solvents used in this study were high-performance liquid chromatography or reagent grade unless otherwise noted.

Preparation of standard solutions. Stock solutions containing 80 μg/ml of each estrogen and stable isotope-labeled estrogen were prepared in methanol containing 0.1% I-ascorbic acid. The stock solutions are stable for at least 2 months while stored at −20° C. Working standard solutions of estrogens at 0.32 and 8 ng/ml were prepared by diluting the stock solutions with methanol containing 0.1% I-ascorbic acid.

Sample preparation. Lung tissue samples (0.1-0.2 g per sample) from Cyp1b1-WT or Cyp1b1-KO mice (female and male, 12-14 weeks of age, n=4-5 per group) were thawed at room temperature, minced with scissors and transferred into 1.5 ml Eppendorf tubes. The tissue was snap-frozen in liquid nitrogen for 5 min, pulverized and transferred into a clean screw-capped glass tube containing 1 ml of ice-cold 12.5 mM ammonium bicarbonate buffer. The tissue was homogenized on ice using a Tissue Tearor™ (Cole-Parmer, Vernon Hills, Ill.) at low and high speeds in two consecutive 15 s increments for a total of 30 s, and further sonicated on ice (five cycles of 10 s pulses with 10 s breaks between pulses). Eight milliliters of ethanol:acetone and 50 μl each of stable isotope-labeled estrogen internal standards (0.32 ng/ml working standard solutions) were added to each tissue homogenate. The mixture was incubated on a rotator at room temperature for 1 h and centrifuged at 3000×g for 30 min. The ethanol:acetone tissue extract was transferred to a clean glass tube and dried under nitrogen gas at 60′C for 60 min (Reacti-Vap III™, Pierce, Rockford, Ill.). The residue was redissolved in 4 ml of methanol, vortexed for 1 min, chilled at −80° C. for 1 h, returned to room temperature and centrifuged at 3000×g for 20 min. The methanolic phase was transferred to a clean glass tube and dried under nitrogen gas. The residue was redissolved in 100 μl of ethanol and vortexed briefly. This step was followed by the addition of 1.5 ml of 100 mM sodium acetate buffer, pH 4.6, and 5 ml of dichloromethane to the residue and incubation at room temperature on a rotator for 30 min.

The extract was chilled at −80° C. for 10 min, returned to room temperature and centrifuged at 3000×g for 20 min. The dichloromethane phase was transferred to a clean tube and dried. To each dried sample, 40 μl of 0.1 M sodium bicarbonate buffer, pH 9.0, and 40 μl of dansyl chloride solution (1 mg/ml in acetone) were added. After vortexing for 10 s, samples were heated at 70° C. (Reacti-Therm III™ Heating Module, Pierce, Rockford, Ill.) for 10 min to form the EM and SI-EM dansyl derivatives. All samples were centrifuged at 3000×g for 20 min and analyzed using LC-MS². The efficiency of extracting estrogen and its metabolites from the tissue cannot be measured accurately because a known amount of each metabolite cannot be placed in the tissue prior to extraction. Furthermore, the amount of estrogens/EM present at baseline is unknown. Use of this same extraction protocol to isolate EM from serum, another highly complex protein mixture, yielded extraction efficiencies ranging from 90 to 105% for the various metabolites.

LC-MS² analysis was performed using a Shimadzu Prominence UFLC system (Shimadzu Scientific Instruments, Columbia, Md.) coupled with a TSQ™ Quantum Ultra triple quadrupole mass spectrometer (Thermo Electron, San Jose, Calif.). The LC separation was carried out on a 50 mm long×2 mm intradermally column packed with 2.5 μm Synergi Hydro-RP particles (Phenomenex, Torrance, Calif.) maintained at 40° C. A 20 μl aliquot of each sample was injected onto the column. The mobile phase, operating at a flow rate of 200 μl/min, consisted of methanol as solvent A and 0.1% (vol/vol) formic acid in water as solvent B. A linear gradient (increasing from 72 to 85% solvent A in 15 min) was employed for the separation. The MS conditions were as follows: source, ESI; ion polarity, positive; spray voltage, 3500 V; sheath and auxiliary gas, nitrogen; sheath gas pressure, 40 arbitrary units; ion transfer capillary temperature, 350° C.; scan type, selected reaction monitoring; collision gas, argon; collision gas pressure, 1.5 mTorr; scan width, 0.7 μm; scan time, 0.01 s; Q1 peak width, 0.70 μm full width at half maximum; Q3 peak width, 0.70 μm FWHM.

Quantitation of tissue estrogens. Quantitation of lung tissue estrogens and EM was carried out using Xcalibur™ Quan Browser (Thermo Electron). Briefly, calibration curves for each EM were constructed by plotting EM-dansyl/SI-EM-dansyl peak area ratios obtained from calibration standards versus amounts of the EM injected on column and fitting these data using linear regression with 1/X weighting. The amounts of EM in the tissue samples were then interpolated using this linear function. The lower limit of quantitation of the analytical method was 0.05 pg EM on column and the lower limit of detection was 5-10 times lower than the lower limit of quantitation.

Quantitative RT-PCR. Total RNA was extracted from frozen lung tissue using TRIzol® Reagent (Life Technologies, Carlsbad, Calif.) according to the manufacturer's instructions. Reverse transcription was carried out using 1 μg RNA and the High Capacity cDNA Kit (Applied Biosystems, Foster City, Calif.). Quantitative PCR reactions were performed on the ABI 7900 instrument using TaqMan® Universal Master Mix and gene-specific primer mixes (both from ABI): Cyp1a1 (Mm00487218_m1), Cyp1b1 (Mm00487229_m1), Comt (Mm01171183_m1) and Hprt (Mm00446968_m1). The Ct values for each gene were normalized to the housekeeping gene Hprt, and the fold change in the transcript level of samples from parallel groups (female versus male, smoke treated versus control) was computed using the comparative Ct method (ΔΔCt; Applied Biosystems Reference Manual, User Bulletin #2).

Statistical analyses. The two-sided Wilcoxon rank sum test was used to compare two groups. The difference was considered significant when the P value was ≤0.05.

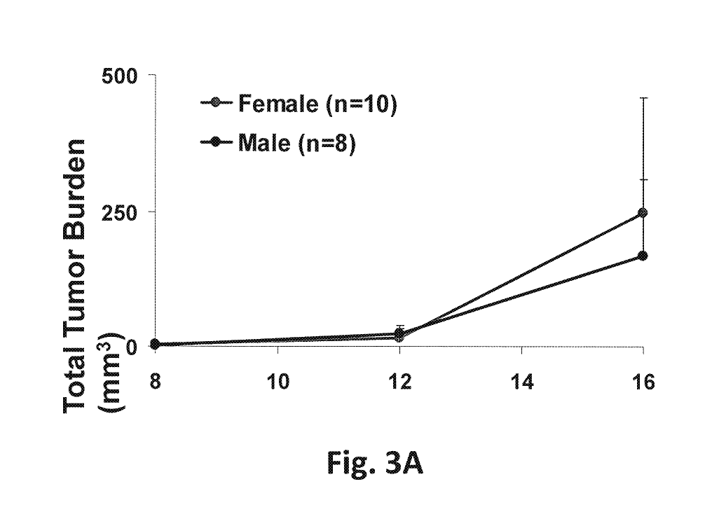

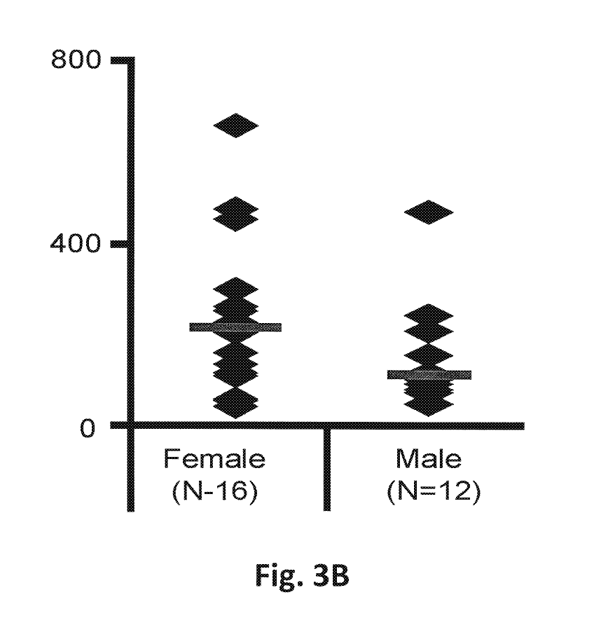

To study lung tumor development, a colony of LSL-KrasG12D mice was established. The LSL-KrasG12D mouse model of conditional lung tumorigenesis carries a latent oncogenic allele of Kras that is often present in human smokers. Intratracheal delivery of adenovirus expressing Cre recombinase (AdeCre) results in activation of Kras only in the lungs. The model recapitulates all stages of human lung cancer progression, from precancerous atypical adenomatous hyperplasia (AAH) to adenoma and adenocarcinoma, with 100% of the animals developing lesions. It was observed that female mice exhibited a 3-fold higher incidence of adenocarcinomas as compared to age-matched males (Table 6). Both the rate of change in total tumor burden (FIG. 3A) and the final tumor burden at 16 weeks of age (FIG. 3B) were increased, although not significantly (p=0.056 p=0.069 respectively), in females as compared to males as measured by magnetic resonance imaging (MRI) over time. These data are consistent with a higher level of 4-OHEs within the lungs of female mice and suggest that 4-OHEs may contribute to lung tumor development, with estrogen metabolites potentially serving as a prognosis marker.

TABLE 6
Incidence of pulmonary lesions in age-
matched AdeCre-infected LSL-Kras^{G12D} mice

| | | |
|---|---|---|
| Lung Cancer Stages | Females | Males |

| | | | |
|---|---|---|---|
| AAH | 15% | (2/12) | 31% (4/13) |
| Adenoma | 23% | (3/13) | 54% (7/13) |
| Adenocarcinoma | 77% | (10/13) | 31% (4/13) |

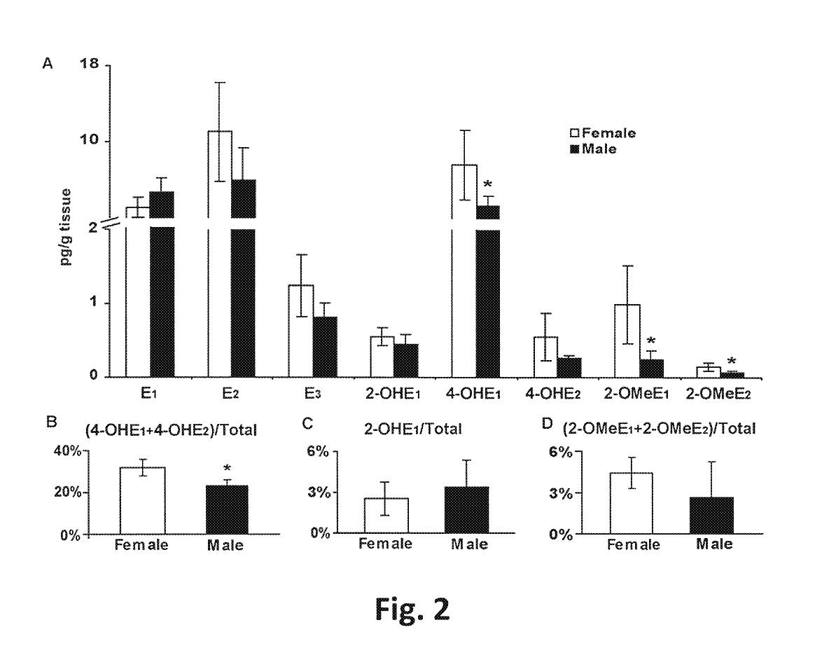

Detection of estrogen and its metabolites in murine lung tissue. Analysis of murine lung tissue has revealed the presence of eight biologically active estrogens/EM within the perfused lungs of male and female wild-type 129SvJ mice. In agreement with previous LC-MS² analyses of lung tissue from A/J mice, E₁ and E₂ were also detected within the lungs of 129SvJ mice. E₃, the predominant estrogen produced during pregnancy, was also found in the lungs of both male and female mice, but at a concentration (^{˜}1 pg/g tissue) much less than that of E₁ (≥3 pg/g) or E₂ (≥6 pg/g) (FIG. 2, panel A). In addition to the three major forms of estrogen, five metabolites of estrogen (2-OHE₁, 4-OHE₁, 4-OHE₂, 2-OMeE₁ and 2-OMeE₂) were detected in murine lung tissue. The levels of 4-OHE₁ within the lungs of both females (7.26 pg/g) and males (3.26 pg/g) were much higher than those of the other metabolites (<1 pg/g). Interestingly, 4-OMeEs were not detected in the murine lung despite the abundance of its precursor 4-OHEs.

Gender differences in the metabolism of estrogen within the murine lung. Distinct differences were observed in the amount of EM within the lungs of male and female mice (FIG. 2). The levels of most EM were higher in the female lung than in the male lung. Both 4-OHE₁ and 4-OHE₂ were 2-fold higher within the lungs of female mice as compared with male mice; the elevation was significant for the more abundant 4-OHE₁ (P=0.032) but not for the 4-OHE₂ metabolite (P=0.094) (FIG. 2, panel A). The concentrations of the putative protective estrogen species, 2-OMeE₁ and 2-OMeE₂, were also higher in female lungs (P=0.008 and P=0.032, respectively). In contrast, the level of 2-OHE₁ was comparable in both genders (FIG. 2, panel A). Even after normalizing for the amount of total estrogen (sum of estrogen and its metabolites) within the lung, the level of 4-OHEs (4-OHE₁ and 4-OHE₂) was 60% higher in the female lung than in the male lung (P=0.016; FIG. 2, panel B). The concentration of neither 2-OHE₁ nor 2-OMeEs varied significantly between genders when expressed as a percentage of total estrogen (FIG. 2, panels C and D).

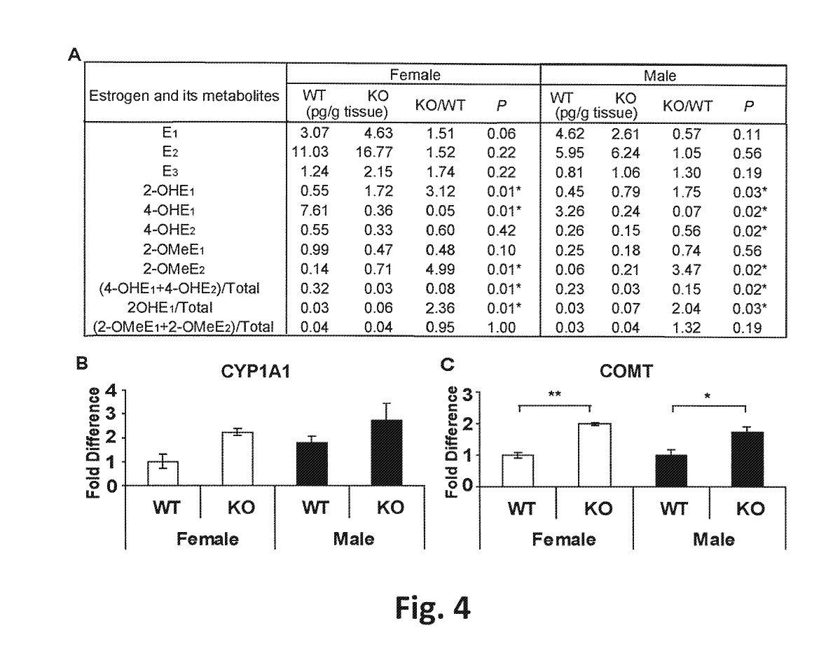

Impact of Cyp1b1 deletion on estrogen metabolism. Because 4-OHEs have been shown to be carcinogenic (26,27), the contribution of the major estrogen-metabolizing enzyme CYP1B1 to the production of 4-OHEs was investigated next by comparing the profile of EM within the lungs of Cyp1b1-WT and Cyp1b1-KO mice. Deletion of Cyp1b1 led to a dramatic decrease in 4-OHE₁ levels in both males (14-fold) and females (21-fold) (7.4 and 4.7% of WT controls, respectively) (FIG. 4, panel A). The level of 4-OHE₂ was reduced by 50% in Cyp1b1-KO mice compared with Cyp1b1-WT controls (56% for males and 60% for females) (FIG. 4, panel A). When expressed as a percentage of total estrogens, 4-OHE levels dropped from 32 (WT) to 3% (KO) in females and from 23 (WT) to 3% (KO) in males (FIG. 4, panel A). These results confirm that 4-OHEs are produced primarily by CYP1B1 in the lung. In contrast with 4-OHEs, the level of 2-OHE₁, the primary metabolite of CYP1A1, was elevated significantly in the lungs of both male and female Cyp1b1-KO mice as compared with WT controls (1.7-fold and 3-fold, respectively). These data suggest that estrogen metabolism is shifted toward 2-hydroxylation in the absence of Cyp1b1. Deletion of Cyp1b1 also increased pulmonary levels of 2-OMeE₂, a product of the major conjugating enzyme COMT, in both males (3.5-fold) and females (5-fold) as compared with WT controls (FIG. 4, panel A).

To determine if increases in the production of 2-OHEs and 2-MeOEs were accompanied by alterations in the expression of Cyp1a1 or Comt, transcript levels were measured in Cyp1b1-WT and Cyp1b1-KO mice by quantitative RT-PCR. The mean level of Cyp1a1 transcripts increased non-significantly in both females and males as a result of Cyp1b1 deletion (P=0.056 and P=0.28, respectively) (FIG. 4, panel B). However, Comt expression was 2-fold higher within the lungs of Cyp1b1-KO mice compared with those of Cyp1b1-WT mice (P=0.008 for females and P=0.016 for males) (FIG. 4, panel C).

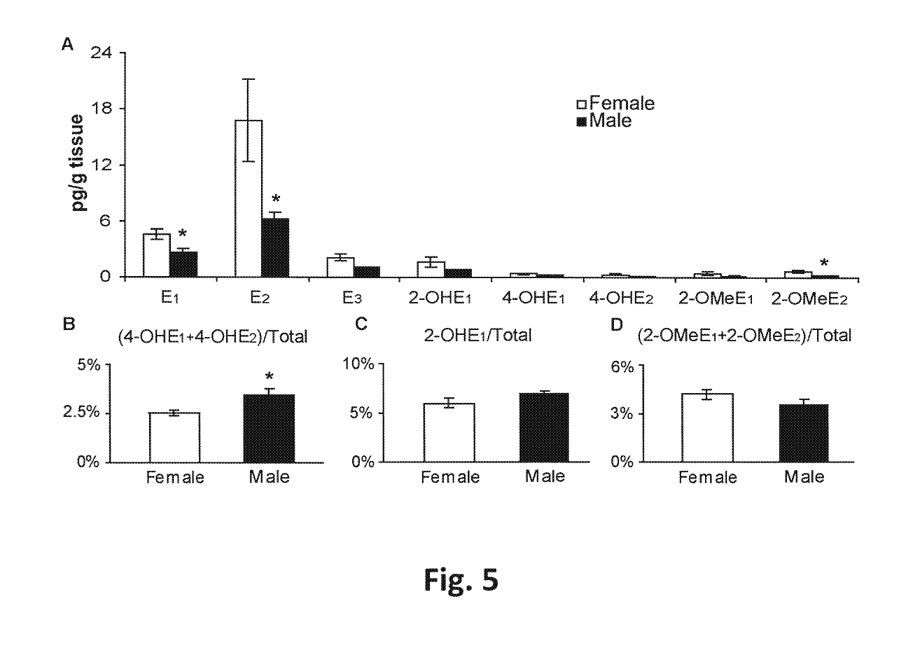

Significant differences in 4-OHE levels between WT males and females were ameliorated by deletion of Cyp1b1 (FIG. 5, panel A). Moreover, 4-OHE levels (percentage of total estrogen) were ^{˜}30% lower in female Cyp1b1-KO mice compared with males (P=0.016) (FIG. 5, panel B). In contrast, 4-OHEs represented a larger percentage of total estrogens in female Cyp1b1-WT mice than in males. Consistent with the findings in Cyp1b1-WT mice, no significant difference was observed in 2-OHE₁ or 2-OMeEs when expressed as a percentage of total estrogen in male and female Cyp1b1-KO mice (FIG. 5, panels C and D).

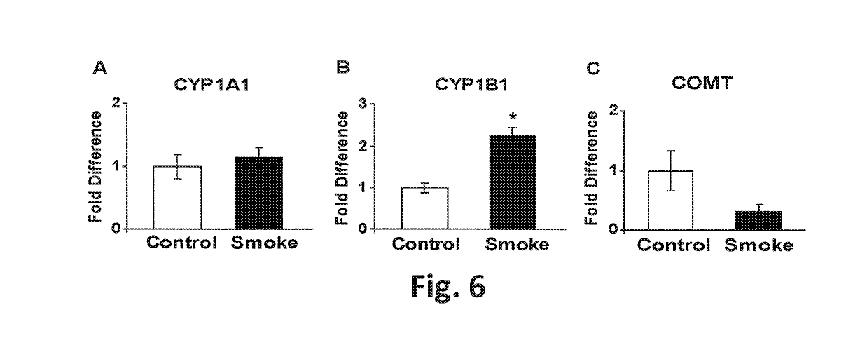

Tobacco smoke modulates pulmonary estrogen metabolism. To extend microarray analysis of tobacco smoke-induced alterations in gene expression, the effect of smoke exposure on the transcript levels of the estrogen-metabolizing genes Cyp1a1, Cyp1b1 and Comt within the lung was examined. Exposure of female C57/B6-APOE*4 mice to tobacco smoke for 8 weeks led to a 2.3-fold increase in Cyp1b1 expression (P=0.008; FIG. 6, panel B). Tobacco smoke also caused a 3.1-fold decrease in the level of Comt mRNA (P=0.095; FIG. 6, panel C). However, no significant change in Cyp1a1 mRNA level was observed following tobacco smoke exposure (FIG. 6, panel A).

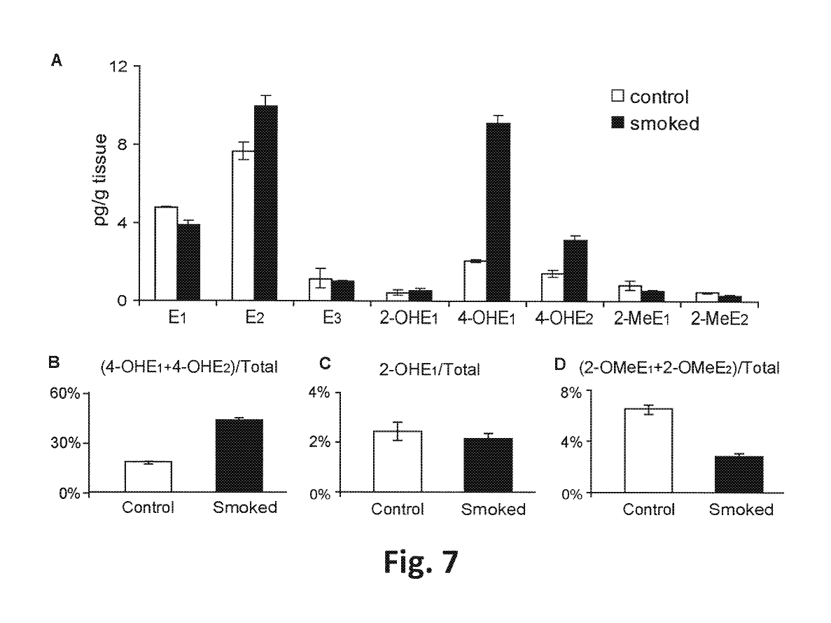

The profile of EM detected within the smoked lung was consistent with the changes in the expression of the estrogen-metabolizing genes that were observed following smoke exposure. Levels of both 4-OHE₁ and 4-OHE₂ were elevated (4- and 2-fold, respectively) in lung tissue from smoke-exposed mice as compared with those of lung tissue from control mice exposed in parallel to filtered air (FIG. 7, panel A). Furthermore, the 4-OHEs (4-OHE₁+4-OHE₂) represented a larger proportion of the total estrogen within the lung following smoke exposure (2-fold higher than that of lungs exposed to filtered air; FIG. 7, panel B). In contrast, 2-OHE1 levels, when expressed either as an absolute value or as a percentage of total estrogen, were not altered by smoke exposure (FIG. 7, panel C). Furthermore, levels of the putative protective EM 2-OMeE₁ and 2-OMeE₂ were decreased to 75 and 71% of control, respectively, in lungs exposed to tobacco smoke (FIG. 7, panel A). This reduction was also reflected in a decrease in 2-OMeEs as a percentage of total estrogen (49% of control; FIG. 7, panel D).

### EXAMPLE 5

### Tobacco Smoke Modulates Estrogen Metabolism in the Human Lung

Estrogen and estrogen metabolites (EMs) were measured in surgically resected lung tumors and adjacent non-neoplastic tissue from female patients with non-small cell lung cancer (NSCLC, 4 never smokers and 5 current smokers) by LC-MS². Current smokers had quit smoking less than one month prior to surgery.

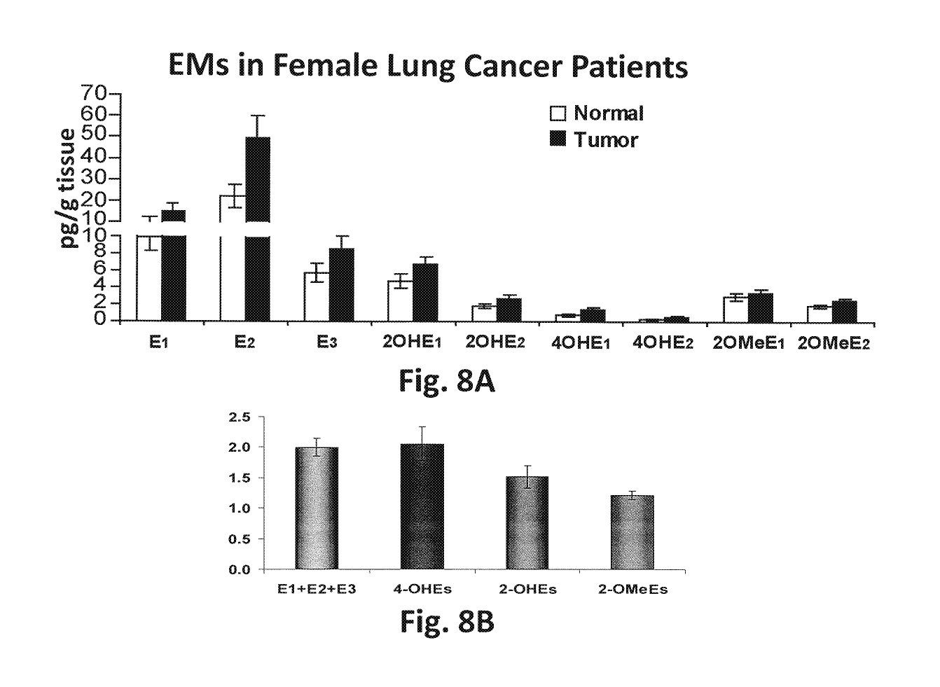

Three estrogens (E₁, E₂ and E₃) and six EMs were detected in human lung tissue. With the exception of one additional metabolite (2-OHE₂), all EMs were identical to those detected previously in the murine lung. All estrogens and EMs were elevated in tumor tissue as compared to adjacent nonneoplastic tissue (p≤0.05 by the signed-rank Wilcoxon test) (FIG. 8A). Levels of total estrogen (E₁+E₂+E₃) and 4-OHEs (4-OHE₁+4-OHE₂) were approximately 2-fold higher in tumor tissue as compared to the adjacent non-neoplastic tissue, while levels of 2-OHEs (2-OHE₁+2-OHE₂) and 2-OMeEs (2-OMeE₁+2 OMeE₂) were increased 1.5 and 1.2-fold, respectively, in tumor tissue (FIG. 8B). These data suggest that estrogen metabolism is altered during lung tumor development in humans.

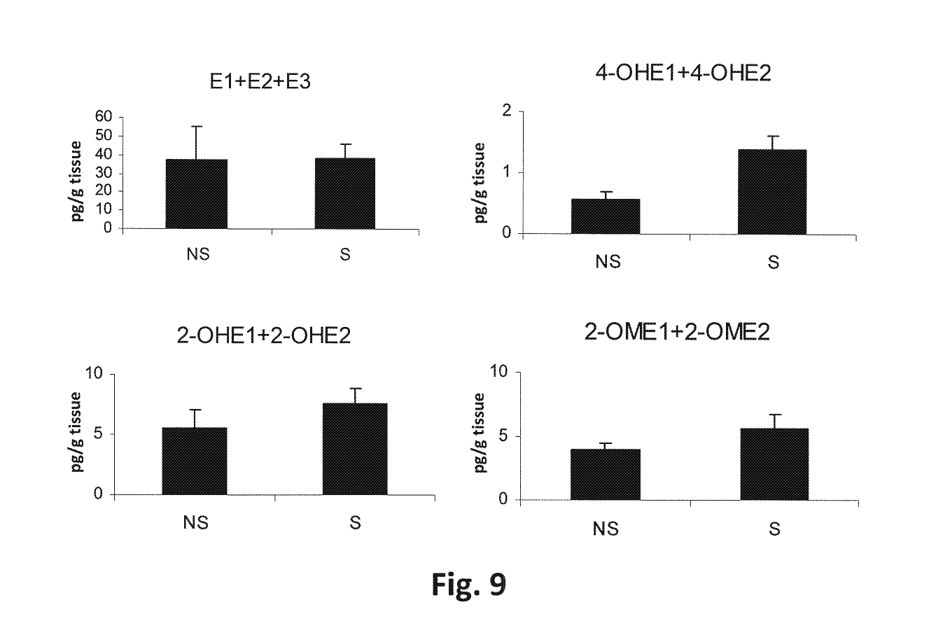

Previous studies from this group indicate that tobacco smoke accelerates the production of 4-OHEs in the mouse lung. To extend this finding, the impact of tobacco smoke exposure on estrogen and EM levels within the human lung was assessed by comparing levels in non-neoplastic lung tissue from current smokers vs nonsmokers. Although levels of estrogen, 2-OHEs and 2-OMeEs were comparable, levels of 4-OHEs were significantly higher in non-neoplastic tissue from current smokers as compared to never smokers (p=0.032 by the Mann-Whitney-Wilcoxon test) (FIG. 9). These data provide additional support for the impact of tobacco smoke on estrogen metabolism within the lung; an interaction that leads to the enhanced production of an estrogen derivative that is known to be carcinogenic (4-OHEs).

The invention is not limited to the embodiments described and exemplified above, but is capable of variation and modification within the scope of the appended claims.

1-110. (canceled)

111. A system for determining a risk of developing lung cancer in a male or female subject, comprising:
a data structure comprising one or more reference concentrations for one or more estrogen metabolites and optionally, one or more reference concentrations for one or more estrogen hormones, wherein the reference concentrations for the one or more estrogen metabolites and the reference concentrations for the one or more estrogen hormones comprise concentrations that indicate a male or female subject is at risk for developing lung cancer, concentrations that indicate the subject has lung cancer, and concentrations that indicate the subject is not at risk for developing lung cancer; and,
a processor operably connected to the data structure, wherein the processor is programmed to compare concentrations of estrogen metabolites determined from a sample obtained from biologic fluid or lung tissue from a subject with the reference concentrations for the one or more estrogen metabolites in the data structure, and optionally is programmed to compare concentrations of an estrogen hormone determined from a sample obtained from biologic fluid or lung tissue from a subject with the reference concentrations for the one or more estrogen hormones in the data structure, and is programmed to generate a lung cancer development risk score based on a comparison of determined estrogen metabolite concentrations with the reference concentrations for the one or more estrogen metabolites in the data structure, and optionally also based on a comparison of determined estrogen hormone concentrations with the reference concentrations for the one or more estrogen hormones in the data structure.

112. The system of claim 111, wherein the one or more estrogen metabolites are selected from the group consisting of 2-OHE₁, 2-OHE₂, 4-OHE₁, 4-OHE₂, 16-alpha-OHE₁, 2-OMeE₁, 2-OMeE₂, 2-hydroxyestrone-3-methyl ester, 4-OMeE₁, 4-OMeE₂, 17-epiestriol, 16-ketoestradiol, and 16-epiestriol.

113. The system of claim 111, wherein the one or more estrogen hormones comprise E₁, E₂, or E₃.

114. (canceled)

115. The system of claim 114, wherein the subject is a human tobacco smoker.

116. The system of claim 115, wherein the human tobacco smoker is a light tobacco smoker.

117. The system of claim 115, wherein the human tobacco smoker is a heavy tobacco smoker.

118-125. (canceled)

126. The system of claim 111, further comprising a second data structure comprising one or more reference nucleic acid sequences having one or more alterations in the CYP1B1 nucleic acid sequence associated with a probability of developing lung cancer caused tobacco smoke exposure, wherein the processor is further programmed to compare a CYP1B1 nucleic acid sequence determined from a sample obtained from a subject with the reference nucleic acid sequences in the second data structure and to generate a lung cancer development risk score based on a comparison of determined estrogen metabolite concentrations with the reference concentrations for the one or more estrogen metabolites in the data structure and also based on a comparison of determined CYP1B1 nucleic acid sequences with the reference nucleic acid sequences in the second data structure, and optionally also based on a comparison of determined estrogen hormone concentrations with the reference concentrations for the one or more estrogen hormones in the data structure.

127. The system of claim 126, wherein the one or more alterations in the CYP1B1 nucleic acid sequence comprise a polymorphism in codon 48 of CYP1B1 DNA, a polymorphism in codon 119 of CYP1B1 DNA, or a polymorphism in codon 432 of CYP1B1 DNA.

128. The system of claim 111, wherein the lung cancer development risk score comprises a high likelihood that the subject will develop lung cancer.

129. The system of claim 126, wherein the lung cancer development risk score comprises a high likelihood that the subject will develop lung cancer.

130. The system of claim 111, further comprising a computer network connection.

131. The system of claim 111, further comprising a computer-readable medium comprising executable code for causing the processor to compare concentrations of estrogen metabolites determined from a sample obtained from a subject with the reference concentrations for the one or more estrogen metabolites in the data structure, and optionally for causing the processor to compare concentrations of an estrogen hormone determined from a sample obtained from a subject with the reference concentrations for the one or more estrogen hormones in the data structure, and to generate a lung cancer development risk score based on a comparison of determined estrogen metabolite concentrations with the reference concentrations for the one or more estrogen metabolites in the data structure, and optionally also based on a comparison of determined estrogen hormone concentrations with the reference concentrations for the one or more estrogen hormones in the data structure.

132. The system of claim 131, wherein the computer-readable medium further comprises executable code for causing the processor to compare a CYP1B1 nucleic acid sequence determined from a sample obtained from a subject with the reference nucleic acid sequences in the second data structure and to generate a lung cancer development risk score based on a comparison of determined estrogen metabolite concentrations with the reference concentrations for the one or more estrogen metabolites in the data structure and also based on a comparison of determined CYP1B1 nucleic acid sequences with the reference nucleic acid sequences in the second data structure, and optionally also based on a comparison of determined estrogen hormone concentrations with the reference concentrations for the one or more estrogen hormones in the data structure.

133. A method for determining a risk of a male or female subject of developing lung cancer, comprising:
comparing the concentration of one or more estrogen metabolites determined from a biologic fluid or lung tissue sample obtained from the subject with a reference concentration of the one or more estrogen metabolites for a healthy subject, a reference concentration of the one or more estrogen metabolites for a subject at risk for developing lung cancer, or a reference concentration of the one or more estrogen metabolites for a subject having lung cancer, using a processor programmed to compare determined concentrations of estrogen metabolites with the reference concentration of the one or more estrogen metabolites for a healthy subject, the reference concentration of the one or more estrogen metabolites for a subject at risk for developing lung cancer, and a reference concentration of the one or more estrogen metabolites for a subject having lung cancer, and
determining whether the subject is healthy, is at risk for developing lung cancer, or has lung cancer based on the comparison.

134. The method of claim 133, wherein the one or more estrogen metabolites are selected from the group consisting of 2-OHE₁, 2-OHE₂, 4-OHE₁, 4-OHE₂, 16-alpha-OHE₁, 2-OMeE₁, 2-OMeE₂, 2-hydroxyestrone-3-methyl ester, 4-OMeE₁, 4-OMeE₂, 17-epiestriol, 16-ketoestradiol, and 16-epiestriol.

135. The method of claim 133, further comprising treating the subject with a regimen capable of improving the prognosis of a lung cancer patient if the subject is determined to have lung cancer.

136. A method, comprising determining the concentration of one or more estrogen metabolites from a tissue sample obtained from a subject, inputting the determined concentration into the system of claim 1, causing the processor of the system to compare the determined concentration of the one or more estrogen metabolites with the reference concentrations for the one or more estrogen metabolites in the data structure, and to generate a lung cancer development risk score based on a comparison of the determined estrogen metabolite concentrations with the reference concentrations for the one or more estrogen metabolites in the data structure.

137. The method of claim 136, wherein the one or more estrogen metabolites are selected from the group consisting of 2-OHE₁, 2-OHE₂, 4-OHE₁, 4-OHE₂, 16-alpha-OHE₁, 2-OMeE₁, 2-OMeE₂, 2-hydroxyestrone-3-methyl ester, 4-OMeE₁, 4-OMeE₂, 17-epiestriol, 16-ketoestradiol, and 16-epiestriol.

138. The method of claim 136, further comprising determining the concentration of one or more estrogen hormones from a tissue sample obtained from the subject, inputting the determined concentration of the one or more estrogen hormones into the system, causing the processor of the system to compare the determined concentration of the one or more estrogen hormones with the reference concentrations for the one or more estrogen hormones in the data structure, and to generate a lung cancer development risk score based on a comparison of the determined estrogen metabolite concentrations with the reference concentrations for the one or more estrogen metabolites in the data structure and also based on a comparison of determined estrogen hormone concentrations with the reference concentrations for the one or more estrogen hormones in the data structure.

139. The method of claim 138, wherein the one or more estrogen hormones comprise E₁, E₂, or E₃.


SEQUENCE LISTING (17 sequences)

SEQ ID NO: 1 - DNA, 18 bp, Artificial Sequence
  Completely synthesized
gctgtctccctctggtta

SEQ ID NO: 2 - DNA, 18 bp, Artificial Sequence
  Completely synthesized
cgttgcagcaggatagcc

SEQ ID NO: 3 - DNA, 18 bp, Artificial Sequence
  Completely synthesized.
gcggaagtgtatcggtga

SEQ ID NO: 4 - DNA, 1630 bp, Homo sapiens
atgggcaccagcctcagcccgaacgacccttggccgctaaacccgctgtccatccagcagaccacgctcctgctactcctgtcggtgctggccactgtgcatgtgggccagcggctgctgaggcaacggaggcggcagctccggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccactgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaat

SEQ ID NO: 5 - DNA, 1630 bp, Homo sapiens
atgggcaccagcctcagcccgaacgacccttggccgctaaacccgctgtccatccagcagaccacgctcctgctactcctgtcggtgctggccactgtgcatgtgggccagcggctgctgaggcaacggaggcggcagctcgggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccactgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaat

SEQ ID NO: 6 - DNA, 1630 bp, Homo sapiens
atgggcaccagcctcagcccgaacgacccttggccgctaaacccgctgtccatccagcagaccacgctcctgctactcctgtcggtgctggccactgtgcatgtgggccagcggctgctgaggcaacggaggcggcagctcgggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccgtccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccactgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaat

SEQ ID NO: 7 - DNA, 1630 bp, Homo sapiens
atgggcaccagcctcagcccgaacgacccttggccgctaaacccgctgtccatccagcagaccacgctcctgctactcctgtcggtgctggccactgtgcatgtgggccagcggctgctgaggcaacggaggcggcagctccggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccagtgaagtggcctaacccggagaactttgatccagctcgattcttggacaaggatggcctcatcaacaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaat

SEQ ID NO: 8 - DNA, 1630 bp, Homo sapiens
atgggcaccagcctcagcccgaacgacccttggccgctaaacccgctgtccatccagcagaccacgctcctgctactcctgtcggtgctggccactgtgcatgtgggccagcggctgctgaggcaacggaggcggcagctccggtccgcgcccccgggcccgtttgcgtggccactgatcggaaacgcggcggcggtgggccaggcggctcacctctcgttcgctcgcctggcgcggcgctacggcgacgttttccagatccgcctgggcagctgccccatagtggtgctgaatggcgagcgcgccatccaccaggccctggtgcagcagggctcggccttcgccgaccggccggccttcgcctccttccgtgtggtgtccggcggccgcagcatggctttcggccactactcggagcactggaaggtgcagcggcgcgcagcccacagcatgatgcgcaacttcttcacgcgccagccgcgcagccgccaagtcctcgagggccacgtgctgagcgaggcgcgcgagctggtggcgctgctggtgcgcggcagcgcggacggcgccttcctcgacccgaggccgctgaccgtcgtggccgtggccaacgtcatgagtgccgtgtgtttcggctgccgctacagccacgacgaccccgagttccgtgagctgctcagccacaacgaagagttcgggcgcacggtgggcgcgggcagcctggtggacgtgatgccctggctgcagtacttccccaacccggtgcgcaccgttttccgcgaattcgagcagctcaaccgcaacttcagcaacttcatcctggacaagttcttgaggcactgcgaaagccttcggcccggggccgccccccgcgacatgatggacgcctttatcctctctgcggaaaagaaggcggccggggactcgcacggtggtggcgcgcggctggatttggagaacgtaccggccactatcactgacatcttcggcgccagccaggacaccctgtccaccgcgctgcagtggctgctcctcctcttcaccaggtatcctgatgtgcagactcgagtgcaggcagaattggatcaggtcgtggggagggaccgtctgccttgtatgggtgaccagcccaacctgccctatgtcctggccttcctttatgaagccatgcgcttctccagctttgtgcctgtcactattcctcatgccaccactgccaacacctctgtcttgggctaccacattcccaaggacactgtggtttttgtcaaccagtggtctgtgaatcatgacccactgaagtggcctaacccggagaactttgatccagctcgattcttggacaaggatggcctcatcagcaaggacctgaccagcagagtgatgattttttcagtgggcaaaaggcggtgcattggcgaagaactttctaagatgcagctttttctcttcatctccatcctggctcaccagtgcgatttcagggccaacccaaatgagcctgcgaaaatgaatttcagttatggtctaaccattaaacccaagtcatttaaagtcaatgtcactctcagagagtccatggagctccttgatagtgctgtccaaaatttacaagccaaggaaacttgccaat

SEQ ID NO: 9 - protein, 543 aa, Homo sapiens
MGTSLSPNDPWPLNPLSIQQTTLLLLLSVLATVHVGQRLLRQRRRQLRSAPPGPFAWPLIGNAAAVGQAAHLSFARLARRYGDVFQIRLGSCPIVVLNGERAIHQALVQQGSAFADRPAFASFRVVSGGRSMAFGHYSEHWKVQRRAAHSMMRNFFTRQPRSRQVLEGHVLSEARELVALLVRGSADGAFLDPRPLTVVAVANVMSAVCFGCRYSHDDPEFRELLSHNEEFGRTVGAGSLVDVMPWLQYFPNPVRTVFREFEQLNRNFSNFILDKFLRHCESLRPGAAPRDMMDAFILSAEKKAAGDSHGGGARLDLENVPATITDIFGASQDTLSTALQWLLLLFTRYPDVQTRVQAELDQVVGRDRLPCMGDQPNLPYVLAFLYEAMRFSSFVPVTIPHATTANTSVLGYHIPKDTVVFVNQWSVNHDPLKWPNPENFDPARFLDKDGLINKDLTSRVMIFSVGKRRCIGEELSKMQLFLFISILAHQCDFRANPNEPAKMNFSYGLTIKPKSFKVNVTLRESMELLDSAVQNLQAKETCQ

SEQ ID NO: 10 - protein, 543 aa, Homo sapiens [sequence omitted]

SEQ ID NO: 11 - protein, 543 aa, Homo sapiens [sequence omitted]

SEQ ID NO: 12 - protein, 543 aa, Homo sapiens
MGTSLSPNDPWPLNPLSIQQTTLLLLLSVLATVHVGQRLLRQRRRQLRSAPPGPFAWPLIGNAAAVGQAAHLSFARLARRYGDVFQIRLGSCPIVVLNGERAIHQALVQQGSAFADRPAFASFRVVSGGRSMAFGHYSEHWKVQRRAAHSMMRNFFTRQPRSRQVLEGHVLSEARELVALLVRGSADGAFLDPRPLTVVAVANVMSAVCFGCRYSHDDPEFRELLSHNEEFGRTVGAGSLVDVMPWLQYFPNPVRTVFREFEQLNRNFSNFILDKFLRHCESLRPGAAPRDMMDAFILSAEKKAAGDSHGGGARLDLENVPATITDIFGASQDTLSTALQWLLLLFTRYPDVQTRVQAELDQVVGRDRLPCMGDQPNLPYVLAFLYEAMRFSSFVPVTIPHATTANTSVLGYHIPKDTVVFVNQWSVNHDPVKWPNPENFDPARFLDKDGLINKDLTSRVMIFSVGKRRCIGEELSKMQLFLFISILAHQCDFRANPNEPAKMNFSYGLTIKPKSFKVNVTLRESMELLDSAVQNLQAKETCQ

SEQ ID NO: 13 - protein, 542 aa, Homo sapiens
MGTSLSPNDPWPLNPLSIQQTTLLLLLSVLATVHVGQRLLRQRRRQLRSAPPGPFAWPLIGNAAAVGQAAHLSFARLARRYGDVFQIRLGSCPIVVLNGERAIHQALVQQGSAFADRPAFASFRVVSGGRSMAFGHYSEHWKVQRRAAHSMMRNFFTRQPRSRQVLEGHVLSEARELVALLVRGSADGAFLDPRPLTVVAVANVMSAVCFGCRYSHDDPEFRELLSHNEEFGRTVGAGSLVDVMPWLQYFPNPVRTVFREFEQLNRNFSNFILDKFLRHCESLRPGAAPRDMMDAFILSAEKKAAGDSHGGGARLDLENVPATITDIFGASQDTLSTALQWLLLLFTRYPDVQTRVQAELDQVVGRDRLPCMGDQPNLPYVLAFLYEAMRFSSFVPVTIPHATTANTSVLGYHIPKDTVVFVNQWSVNHDPLKWPNPENFDPARFLDKDGLISKDLTSRVMIFSVGKRRCIGEELSKMQLFLFISILAHQCDFRANPNEPAKMNFSYGLTIKPKSFKVNVTLRESMELLDSAVQNLQAKETC

SEQ ID NO: 14 - DNA, 23 bp, Artificial sequence
  Completely synthesized
aaatcaaaacagatacccggatg

SEQ ID NO: 15 - DNA, 18 bp, Artificial sequence
  Completely synthesized.
tccggcctctcacttgca

SEQ ID NO: 16 - DNA, 20 bp, Artificial sequence
  Completely synthesized.
tgaatgaactgcaggacgag

SEQ ID NO: 17 - DNA, 20 bp, Artificial sequence
  Completely synthesized.
acgacttgggcttaatggtc